(19) United States
(12) Patent Application Publication
(10) Pub. No.: US 2023/0054852 A1
(43) Pub. Date: Feb. 23, 2023

(54) p53 POST-TRANSLATIONAL MODIFICATIONS AS MARKERS IN THE DIAGNOSIS AND PROGNOSIS OF A NEURODEGENERATIVE DISEASE

(71) Applicant: Diadem S.r.l., Brescia (IT)

(72) Inventors: Simona Piccirella, Brescia (IT); Daniela Letizia Uberti, Brescia (IT)

(21) Appl. No.: 17/699,030

(22) Filed: Mar. 18, 2022

Related U.S. Application Data
(63) Continuation-in-part of application No. 17/398,815, filed on Aug. 10, 2021.
Application No. 17/398,815 is a continuation of application No. PCT/IB2021/056792, filed on Jul. 27, 2021.

(30) Foreign Application Priority Data
Jul. 30, 2020 (IT) 102020000018544

(51) Int. Cl. G01N 33/68

(52) U.S. Cl. CPC G01N 33/6896; G01N 33/6818; G01N 2800/2821

(57) ABSTRACT

The present invention refers to p53 sequence and post translational modifications (PTMs) and to their use as biomarkers in the diagnosis of neurodegenerative disease and cognitive decline and/or in the prognosis of Alzheimer's disease at different stages and/or of neurodegenerative disease in a biological sample. The invention also provides for a 1) diagnostic method based on a highly accurate mass spectrometry analysis for the diagnosis of neurodegenerative disease, including Mild Cognitive Impairment (MCI), Alzheimer's disease (AD), fronto-temporal dementia (FTD), Lewi's Body (LB), and vascular dementia (VD) in a subject, by evaluating the PTMs to the said p53 linear sequence protein and possible cut of its full sequence specifically in human plasma of patients; and 2) prognosis of AD in CU and MCI patients.

### CROSS REFERENCE TO RELATED APPLICATIONS

The present application is a continuation in part of and claims priority to U.S. patent application Ser. No. 17/398,815, entitled “p53 post-translational modifications as markers in the diagnosis and prognosis of a neurodegenerative disease, ” filed on Aug. 10, 2021, which is a continuation of and claims priority to International Application No. PCT/IB2021/056792, entitled “p53 Post-Translational Modifications as Markers in the Diagnosis and Prognosis of a Neurodegenerative Disease,” filed on Jul. 27, 2021, which claims priority to and the benefit of Italian Patent Application No. 102020000018544, entitled “p53 Post-Translational Modifications as Markers in the Diagnosis and Prognosis of a Neurodegenerative Disease,” filed on Jul. 30, 2020, the disclosures of which are incorporated herein by reference in their entireties.

### SEQUENCE LISTING

This application contains a Sequence Listing which has been submitted electronically in ASCII format and is hereby incorporated by reference in its entirety. Said ASCII copy, created on Mar. 18, 2022, is named 121689-10305_sequence_ST25.txt and is 20.790 bytes in size.

### FIELD OF THE INVENTION

The present invention refers to p53 sequence and post translational modifications (PTMs) and to their use as biomarkers in the diagnosis of a neurodegenerative disease and cognitive decline to Alzheimer's disease and Alzheimer's disease and/or in the prognosis of Alzheimer's disease at different stages and/or of neurodegenerative disease in a biological sample. The invention also provides for a diagnostic method based on a highly accurate mass spectrometry analysis for the diagnosis of neurodegenerative disease, including Mild Cognitive Impairment (MCI), Alzheimer's disease (AD), fronto-temporal dementia (FTD), Lewi's Body (LB), and vascular dementia (VD) in a subject, by evaluating the changes (PTMs) to said p53 linear protein sequence specifically in a biofluid sample. The invention also provides for a diagnostic method based on a highly accurate mass spectrometry analysis for the prognosis of Alzheimer's disease (AD) at asymptomatic and prodromal stages (MCI) by evaluating the changes of said PTMs to the linear sequence of p53 protein specifically in a biofluid sample. The invention also provides a method of selecting a treatment based on the early diagnosis of neurodegenerative disease and a method to monitor the progress of such treatment of neurodegenerative disease.

### BACKGROUND

The confirmation of the presence of a large amount of altered conformational p53 isoform as an early risk factor for Alzheimer's disease (shortly ‘AD’) have been demonstrated in different published studies [1-3]. Initially, more than 400 subjects among AD, Mild Cognitive Impairment, Parkinson Disease, other Dementia and healthy subjects were enrolled in different independent studies and tested for Unfolded p53 by using different techniques (immunoprecipitation experiments, FACS analysis, ELISA) with a commercial conformational specific anti-p53 antibody [4-7]. In 2006 for the first time Uberti et al. [8], demonstrated that fibroblasts from sporadic Alzheimer's disease (AD) patients specifically expressed an anomalous and detectable conformational state of p53 that differentiate these cells from fibroblasts of age-matched non-AD subjects. In this conformational altered state, p53 lost its ability to transactivate its target genes, and consequently its biological functions [9-10]. The higher amount of unfolded p53 was also confirmed in blood of AD compared to healthy-non demented subjects or patients affected by other dementia and PD, as well as in MCI converted to AD. Altogether these data suggested a direct association between Unfolded p53 and AD pathology.

In EP3201234B1, it has been reported the development of a new conformational specific anti-Up53 antibody named 2D3A8, that binds to an epitope (aa 282-297), accessible only when p53 loses its wild type conformation towards an unfolded phenotype. Comparing to the commercial antibody used at the beginning of Unfolded p53 discovering in AD (PAb240, aa214-217), the 2D3A8 antibody showed higher sensitivity and specificity in identifying AD patients compared to healthy elderly in Oviedo cohort.

In particular, said immunodiagnostic method is able to identify immunocomplex in a biological sample that are indicative of AD and to determine the predisposition of a subject affected by Mild Cognitive Impairment (MCI) to develop AD.

PCT/IB2019/051785 discloses a method based on the identification and quantification of the levels of specific p53 peptides, indicated as “P 1” and “P2”, that have been detected by mass spectrometry analysis in human plasma of patients affected by Alzheimer's disease or patients that have symptoms that can predispose to the development of AD.

There is now the need of identifying new specific biological markers that can be used in the diagnosis and/or prognosis of Alzheimer's disease and of developing an accurate and sensible diagnostic method that can be used for the diagnosis and/or prognosis of AD, in particular at the pre-clinical and prodromal stages of the disease and for the differential analysis of AD from other forms of dementia, such as Frontotemporal Dementia, Levy Body dementia and vascular dementia.

Many of the clinical trials for assessing the efficacy of a drug for its potential to treat specific neurodegenerative disease state end in failure because the patient population being assessed is heterogenous, as they often have a mixture of patients having different neurodegenerative disease states and won't respond if they are being treated with a drug that is effective for one disease state and not the other. Therefore, there is a need to properly stratify the patient population to ensure that the patients being treated are homogenous having the same disease state when being assessed for drug efficacy.

### SUMMARY OF THE INVENTION

The object of the present invention has been achieved by identifying eleven main post-translation modifications (PTMs) in the amino acidic sequence of the p53 protein within the region of amino acids 1-371, herein called PTM-1, PTM- 2, PTM-3, PTM- 4, PTM-5, PTM-6, PTM-7, PTM-8, PTM-9, PTM-10, PTM-11 and/or some truncated forms of the p53 protein in a biofluid sample.

An aspect of the present invention therefore relates to a diagnostic method based on the identification of said PTMs for use in the diagnosis of different forms of dementia and cognitive decline and/or in the prognosis of Alzheimer's disease at different stages.

An aspect of the present invention relates to a method of treatment of neurological diseases comprising the steps of identification of said PTMs for use in the diagnosis of specific disease state and treating the disease state with suitable drugs specific for each indication.

The invention therefore includes a method of treating a subject who presents with one or more symptoms of a neurodegenerative disease recited herein, comprising: identifying a subject having the recited disease by determining the presence and/or absence of one or more post-translational markers (PTMs) of PTMs 1-12 in a biofluid sample obtained from the subject, and treating the subject so-identified with a drug that is suitable for treatment of the recited neurodegenerative disease.

The invention also includes a method for confirming diagnosis of a neurodegenerative disease as set forth herein in a subject diagnosed with the specific disease, comprising identifying the presence and/or absence of at least one PTM selected from PTMs 1-12, as described herein, in a biofluid sample from the subject so-diagnosed, thereby indicating the neurodegenerative disease corresponding to the presence and/or absence of the at least one PTM of PTMs 1-12, and wherein when the specified presence and/or absence of the recited PTMs is identified in the biofluid sample, the diagnosis of the specified neurodegenerative disease is confirmed in the subject.

An aspect of the present invention relates to a method of assessing the efficacy of a drug for treating a specific neurological disease comprising the steps of identification of PTMs indicating a given neurodegenerative disease, and treating the disease with said drug and monitoring the progress or remission of disease state from one to another.

An in vitro method of identifying a drug for treatment of a given neurodegenerative disease as set forth herein may comprise: providing (i) a biofluid sample obtained from a subject having symptoms of the neurodegenerative disease prior to treatment with a drug, and (ii) a biofluid sample obtained from said subject following treatment with the drug, wherein said subject has been determined to have the given neurodegenerative disease by identifying the presence and/or absence of at least one of PTMs 1-12, as set forth herein, and wherein a comparison of PTMs present and/or absent in sample (ii) relative to sample (i) indicates that the drug is useful or potentially useful to treat the neurodegenerative disease in a subject.

The method for identifying a drug effective to treat a given neurodegenerative disease may further comprise formulating the drug so-identified into a medicament.

An aspect of the present invention relates to a method of assessing the efficacy of a combination or a cocktail of drugs for treating a specific neurological disease comprising the steps of identification of said PTMs, identifying the disease state, treating the disease with said combination and monitoring the progress or remission of disease state from one to another.

An aspect of the present invention relates to a method of assessing the efficacy of a plurality of drugs for treating a specific neurological disease comprising the steps of identification of said PTMs, identifying the disease state, treating the disease with a first drug and monitoring the progress or remission of disease state from one to another, treating the disease with a second drug and monitoring the progress or remission of disease state from one to another, treating the disease with said first and second drug and monitoring the progress or remission of disease state from one to another.

An aspect of the present invention relates to a method of stratifying patient population for assessing the efficacy of a drug being evaluated for a specific neurodegenerative disease state comprising the steps of identification of said PTMs, identifying the disease state, stratifying the patient according to the specific disease state, isolating the patient having a specific disease state and treating the disease with said drug.

### BRIEF DESCRIPTION OF THE DRAWINGS

The characteristics and the advantages of the present invention will become apparent from the following detailed description and the working examples provided for illustrative purposes, as well as the annexed Figures, wherein:

FIG. 1. Protein ubiquitination sites detected in samples of subjects affected by AD.

FIG. 2. Protein ubiquitination sites detected in control samples (cognitively unimpaired (CU)).

FIG. 3. Protein ubiquitination sites detected in the samples of subjects affected by frontal dementia (FTD).

FIG. 4. Protein ubiquitination sites detected in the samples of subjects affected by Lewy Body's dementia (LB).

FIG. 5. Protein ubiquitination sites detected in the samples of subjects affected by vascular dementia (VD).

FIG. 6. Protein ubiquitination sites detected in the samples of subjects affected by mild cognitive disorder (MCI).

FIG. 7. Protein ubiquitination sites detected in the samples of cognitively healthy subjects (CU) who developed AD over a period of at least 18 months.

FIG. 8. Protein ubiquitination sites detected in the samples of the subjects of the AD developed MCI group.

FIG. 9. The position of PTMs1-11 and truncation in the N-terminal region within the amino acids 1-248 of SEQ ID NO: 1, identified here as PTM-12, in the p-53 protein is shown

The sequences reported in the figures correspond to the linear sequence of SEQ IDN: 1.

### DEFINITIONS

With the term “U-p53” it is meant to denote the region of amino acids 1-371 of the p53 protein, which involves the post translational modifications (PTMs), and in some cases also a truncation, on linear protein sequence as described below.

With term “p53” it is meant the wild-type protein p53 as following the Database “UniProtKB, Protein ID: P04637, amino acids: 1-393”.

With the term “neurodegenerative disease” it is meant to denote a range of conditions that mainly affect the neurons in the human brain, also comprising forms of dementia, such as Mild Cognitive Impairment (MCI), fronto-temporal dementia (FTD), Lewi's Body (LB), and vascular dementia (VD), as well as the different stages of the said neurodegenerative diseases and cognitive decline to dementia, and Alzheimer's disease (AD) (including pre-clinical and prodromal stages).

### DETAILED DESCRIPTION OF THE INVENTION

The invention therefore relates to a combination of p53 post translational modifications detected by a highly accurate mass spectrometry method that can be used as biomarkers in an in vitro o ex vivo method for the diagnosis of a neurodegenerative disease. Said method is based on the identification of specific p53 modifications compared to its linear sequence, shorty referred to as ‘PTMs’, that have been detected by mass spectrometry analysis in a biofluid sample derived from patients affected by Alzheimer's disease or patients that have symptoms that can predispose to the development of AD or to different forms of dementia.

In particular, first, p53 protein is captured by immunoprecipitation in a biofluid sample from patients at pre-clinical, prodromal clinical stages of Alzheimer's, Mild Cognitive Impairment (MCI) stable patients, and cognitive unimpaired subjects (CU), Frontotemporal Dementia (FD), Vascular Dementia (VD) and Lewy Body Dementia (LB). Then, the post translational modifications of the captured protein are identified by protein sequencing with a highly sensitive selective mass spectrometry method. After sequencing, the post translational modifications are also identified by a database searching to check ones already described in literature.

The data obtained for each sample are then compared with PTMs detected in the biofluid samples from subjects with same clinical evidence showing a correlation between “PTMs and diagnosis”, therefore demonstrating a strong evidence that the U-p53 PTMs can be considered as highly reliable biomarkers in the prognosis and diagnosis of a neurodegenerative disease.

Said method is advantageously fast, requires a small volume of biofluid sample and reliably identifies U-p53 PTMs in each sample analysed.

Furthermore, the method and the biomarkers identified can be used also in the diagnosis and prognosis of Alzheimer's disease in asymptomatic individuals and people suffering from MCI, thus allowing the access to the diagnostics market.

Furthermore, the method and the biomarkers identified can be used also for differentiating Alzheimer's disease, from other forms of dementia, such as LB, VD, FTD in demented patients. In fact, as it will be seen below, the U-p53 protein sequence in biofluid samples of patients affected by Alzheimer's disease shows a variability in terms of length within the region of amino acids 1-271, said variability including a truncation within the same region. It should be appreciated that said variability and truncation are peculiar of Alzheimer's disease, as the same are not detected in biofluid samples of patients affected by other forms of dementia, much less in cognitive unimpaired subjects.

In some embodiments, the presence of truncation in the region denoted by the amino acids 1-248 of SEQ ID NO: 1 indicates the presence of Alzheimer's disease (AD). In some embodiments, the presence of an additional truncation in the region denoted by the amino acids 249-271 of SEQ ID NO: 1 indicates the presence of Alzheimer's disease (AD). The additional truncation in the region 249-271 is seen as variability in the length of the peptides being generated. See Table 2. At the same time, a residual amount of U-p53 in the biofluid samples keeps its sequence length, whereon peculiar PTMs of Alzheimer's disease are detected. It follows that patients affected by Alzheimer's disease are unequivocally identified and distinguished from other dementia patients, insofar as the former show both a truncation in the U-p53 protein sequence and peculiar PTMs in the residual amount of untruncated U-p53 protein

In addition, since said biomarkers can be used in the prognosis of cognitive decline to Alzheimer's Dementia in asymptomatic and MCI subjects and in the diagnosis of neurodegenerative disease as the dementia, said method advantageously allows the use of a U-p53 PTMs to select the subjects in clinical trials to enable success of the trial and to differentiate patients affected by AD from other forms of dementia as LB, VD, FTD.

The present invention thus relates to an in vitro or ex vivo method for the diagnosis or prognosis of a neurodegenerative disease, the method comprising the steps of:

a) analysing a biofluid sample for the presence of post-translation modifications (PTMs) in the region of amino acids 1-371 of the p53 protein (U-p53), said PTMs being:
PTM-1 at the amino acid M1,
PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33,

wherein the presence of at least two PTMs selected from PTM-2, PTM-7, PTM-8, and PTM-11 is indicative of a cognitive unimpaired subject (CU),

b) assessing the presence of:
at least two PTMs selected from PTM-1, PTM-3, PTM-4, PTM-5, PTM-6, PTM-9, and PTM-10, and
at least one PTM selected from PTM-2, PTM-7, PTM-8, and PTM-11, as indicative of the occurrence or the risk of development of a neurological disease, said neurodegenerative disease being selected from Mild Cognitive Impairment (MCI), Alzheimer's disease (AD), Fronto-temporal dementia (FTD), Lewi's Body (LB), and vascular dementia (VD),

c) correlating the PTMs assessed in step b) with those identifying the corresponding neurodegenerative disease.

According to the present invention, preferably in the in vitro or ex vivo method:

the post-translation modification PTM-1 has a group CO—CH₃ branched to the amino acid M1 of the p53 protein;

the post-translation modification PTM-2 has a group CO—CH₃ branched to the amino acid K164 of the p53 protein;

the post-translation modification PTM-3 has a group CO—CH₃ branched to the amino acid K370 of the p53 protein;

the post-translation modification PTM-4 has a ubiquitination site [GG] branched at the amino acid K101 of the p53 protein;

the post-translation modification PTM-5 has a ubiquitination site [GG] branched at the amino acid K120 of the p53 protein, where [GG] denotes a lateral chain of two residues of “Glycine”;

the post-translation modification PTM-6 has a ubiquitination site [GG] branched at the amino acid K132 of the p53 protein;

the post-translation modification PTM-7 has a ubiquitination site [GG] branched at the amino acid K139 of the p53 protein;

the post-translation modification PTM-8 has a ubiquitination site [GG] branched at the amino acid K291 of the p53 protein;

the post-translation modification PTM-9 has a ubiquitination site [GG] branched at the amino acid K357 of the p53 protein;

the post-translation modification PTM-10 has phosphorylation at the amino acid S6 of the p53 protein;

the post-translation modification PTM-11 has phosphorylation at the amino acid S33 of the p53 protein.

In a preferred embodiment, the in vitro or ex vivo method of the present invention is for differentiating Alzheimer's disease, from other forms of dementia, such as LB, VD, FTD in demented patients.

In fact, as said above, the assessment of following criteria are indicative of AD:

a sequence variability in terms of length within the region of amino acids 1-271, said variability including a truncation within the same region, and the presence of at least two

PTMs selected from PTM-1, PTM-3, PTM-4, PTM-5, and PTM-6, in a residual amount of untruncated sequence, preferably the presence of all PTM-1, PTM-3, PTM-4, PTM-5, and PTM-6.

Said truncation, mainly due to biological reactions, does not affect the detectability of PTMs in said residual amount of untruncated sequence.

In some embodiments, the presence of truncation in the region denoted by the amino acids 1-248 of SEQ ID NO: 1 and presence of at least one of PTMs-1, 2, 3, 4, 5 and 6 indicate that the patient is affected by AD. Therefore one can distinguish the presence of AD from CU or MCI in a patient by identifying the PTM fingerprint which comprises of said truncation and presence of at least one of PTMs-1, 3, 4, 5 and 6.

In some embodiments, the presence of truncation in the region denoted by the amino acids 1-248 of SEQ ID NO: 1 and presence of at least one of PTMs-1, 2, 3, 4, 5 and 6 and the absence of at least one of PTM-7, PTM-9 and PTM-10 indicate that the patient is affected by AD. Therefore one can distinguish the presence of AD from CU, MCI, FTD, LB or VD in a patient by identifying the specific PTM fingerprint of AD.

An in vitro or ex vivo method to differentiate Alzheimer's Disease (AD) from Cognitive Unimpaired (CU) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12,

wherein AD is indicated by:

(a) the presence of a truncation of the region denoted by amino acids 1-248 of p53 protein (SEQ ID NO:1),

(b) the presence of at least one of PTMs-1, 3, 4, 5 and 6, and

(c) the absence of PTM-7;

and

wherein CU is indicated by:
(a) the presence of PTM-7,
(b) no truncation of the region denoted by amino acids 1-248 of p53 protein (SEQ ID NO:1), and
(c) the absence of at least one of PTMs-1, 3, 4, 5, and 6,

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

As used herein, at least one of refers to one, at least two of or two, at least three of or three, at least four of or four, at least five of or five, at least six of or six, at least seven of or seven at least eight of or eight, at least nine of or nine, at least ten of or ten, at least eleven of or eleven, and twelve of.

An in vitro or ex vivo method to differentiate Alzheimer's Disease (AD) and a neurodegenerative disease selected from the following: Mild Cognitive Impairment (MCI), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein AD is differentiated from a said neurodegenerative diseases as follows (A)-(D), respectively:

(A) AD differentiated from MCI:

wherein AD is indicated by:
(a) a truncation of the region denoted by amino acids 1-248 of p53 protein (SEQ ID NO:1), and
(b) the presence of at least one of PTMs-2, 3, 4, 5, and 6, and
(c) the absence of at least one of PTMs-7 and 10; and wherein MCI is indicated by:
(a) the presence of at least one of PTMs-7 and 10, and
(b) no truncation of the region denoted by amino acids 1-248 of p53 protein (SEQ ID NO:1), and
(c) the absence of at least one of PTMs-2, 3, 4, 5, and 6;

(B) AD differentiated from FTD:

wherein AD is indicated by the presence of:
(a) a truncation of the region denoted by amino acids 1-248 of p53 protein (SEQ ID NO:1), and
(b) the presence of at least one of PTMs-1, 2, 3, 4, 6 and 11, and
(c) the absence of PTM-9;

wherein FTD is indicated by:
(a) the presence of PTM 9,
(b) no truncation of region denoted by amino acids 1-248 of p53 protein (SEQ ID NO:1), and
(c) the absence of at least one of PTMs-1, 2, 3, 4, 6 and 11;

(C) AD differentiated from LB:

wherein AD is indicated by the presence of:
(a) a truncation of the region denoted by amino acids 1-248 of p53 protein (SEQ ID NO:1), and
(b) the presence of at least one of PTMs-1, 3, 4 and 11;

wherein LB is indicated by:
(a) no truncation of the region denoted by amino acids 1-248 of p53 protein (SEQ ID NO:1), and
(b) the absence of at least one of PTMs-1, 3, 4, and 11;

and

(D) AD differentiated from VD:

wherein AD is indicated by the presence of:
(a) a truncation of the region denoted by amino acids 1-248 of p53 protein (SEQ ID NO:1), and
(b) the presence of at least one of PTMs-1, 3, 4 and 11;

wherein VD is indicated by:
(a) no truncation of the region denoted by amino acids 1-248 of p53 protein (SEQ ID NO:1), and
(b) the absence of at least one of PTMs 1, 2, 3, 6 and 11,

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

An in vitro or ex vivo method to differentiate Cognitive Unimpaired (CU) and a neurodegenerative disease selected from the following: Mild Cognitive Impairment (MCI), MCI with a prognosis of cognitive decline of AD (MCI to AD), cognitive decline of an asymptomatic AD (CU to AD), Alzheimer's disease (AD), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein CU is differentiated from a said neurodegenerative diseases as follows (A)-(G), respectively:

(A) CU is differentiated from MCI:

wherein CU is indicated by:
(a) the presence of PTM-2,
(b) the absence of at least one of PTM1 and PTM10;

and wherein MCI is indicated by:
(a) the presence of at least one of PTM-1, and PTM-10, and
(b) the absence of PTM-2;

(B) CU is differentiated from MCI to AD:

wherein CU is indicated by:
(a) the presence of PTM-2, and
(b) the absence of at least one of PTM-1, PTM-3, PTM-5, PTM6, and PTM-10;

and wherein MCI to AD is indicated by:
(a) the presence of at least one of PTM-1, PTM-3, PTM-5, PTM-6 and PTM-10, and
(b) the absence of PTM-2

(C) CU is differentiated from CU to AD:

wherein CU is indicated by:
(a) the presence of at least one of PTM-2, 7, 8, and 11
(b) the absence of at least one of PTM-4 and 5,
(c) no truncation of the region denoted by amino acids 1-248 of p53 protein (SEQ ID NO:1)

wherein CU to AD is indicated by:
(a) the presence of at least one of PTM-4, and PTM-5,
(b) the absence of at least one of PTM-2, PTM-7, PTM-8 and PTM-11;

(D) wherein CU is differentiated from AD:

wherein CU is indicated by:
(a) the presence of PTM-7,
(b) the absence of PTM-1 and PTM3-6, and
(c) no truncation of the region denoted by amino acids 1-248 of p53 protein (SEQ ID NO:1)

wherein AD is indicated by:
(a) the presence of at least one of PTM-1, and PTM3-6,
(b) the absence of PTM-7, and
(c) truncation of the region denoted by amino acids 1-248 of p53 protein (SEQ ID NO:1);

(E) wherein CU is differentiated from FTD:

wherein CU is indicated by:
(a) the presence of at least one of PTM-2, PTM-7 and 11,
(b) the absence of at least one of PTM-5 and PTM-9;

wherein FTD is indicated by:
(a) the presence of at least one of PTM-5 and PTM-9, and
(b) the absence of at least one of PTM-2, PTM-7 and PTM-11;

(F) wherein CU is differentiated from LB:

wherein CU is indicated by:
(a) the presence of at least one of PTM-7 and PTM-11, and
(b) the absence of at least one of PTM-5 and PTM-6;

wherein LB is indicated by:
(a) the presence of at least one of PTM-5 and PTM-6, and
(b) the absence of at least one of PTM-7 and PTM-11;

(G) wherein CU is differentiated from VD:

wherein CU is indicated by:
(a) the presence of at least one of PTM-2, PTM-7 and PTM-11, and
(b) the absence of at least one of PTM-4 and PTM-5;

wherein VD is indicated by:
(a) the presence of at least one of PTM-4 and PTM-5, and
(b) the absence of at least one of PTM-2, PTM-7 and PTM-11;

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

An in vitro or ex vivo method to differentiate Mild Cognitive Impairment (MCI) from a neurodegenerative disease selected from the following:, MCI with a prognosis of cognitive decline of AD (MCI to AD), cognitive decline of an asymptomatic AD (CU to AD), Alzheimer's disease (AD), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein MCI is differentiated from a said neurodegenerative disease as follows (A)-(F):

(A) MCI is differentiated from MCI to AD:

wherein MCI is indicated by:
(a) the absence of at least one of PTM-3, PTM-5 and PTM-6,

wherein MCI to AD is indicated by:
(a) the presence of at least one of PTM-3, PTM-5 and PTM-6;

(B) MCI is differentiated from CU to AD:

wherein MCI is indicated by:
(a) the presence of at least one of PTM-1, PTM-7, PTM-10 and PTM-11, and
(b) the absence of at least one of PTM-4, PTM-5 and PTM-9;

wherein CU to AD is indicated by:
(a) the presence of at least one of PTM-4, PTM-5 and PTM-9,
(b) the absence of at least one of PTM-1, PTM-7, PTM-10 and PTM-11;

(C) MCI is differentiated from AD:

wherein MCI is indicated by:
(a) the presence of at least one of PTM-7, PTM-8 and PTM-10,
(b) the absence of at least one of PTM-2 to PTM-6, and
(c) no truncation of the region denoted by amino acids 1-248 of p53 protein (SEQ ID NO:1);

wherein AD is indicated by:
(a) the presence of at least one of PTM-2 to PTM-6,
(b) the absence of at least one of PTM-7, PTM-8 and PTM-10, and
(c) truncation of the region denoted by amino acids 1-248 of p53 protein (SEQ ID NO:1);

(D) MCI is differentiated from FTD:

wherein MCI is indicated by:
(a) the presence of at least one of PTM-1, PTM7 , PTM-10 and PTM-11
(b) the absence of at least one of PTM-5 and PTM-9;

wherein FTD is indicated by:
(a) the presence of at least one of PTM-5 and PTM-9, and
(b) the absence of at least one of PTM-1, PTM7 , PTM-10 and PTM-11;

(E) MCI is differentiated from LB:

wherein MCI is indicated by:
(a) the presence of at least one of PTM-1, PTM-7, PTM-10 and PTM-11,
(b) the absence of at least one of PTM-2, and PTM-4 to PTM-6

wherein LB is indicated by:
(a) the presence of at least one of PTM-2, PTM-4, PTM5 and PTM-6
(b) the absence of at least one of PTM-1, PTM-7, PTM-10 and PTM-11;

(F) MCI is differentiated from VD:

wherein MCI is indicated by:
(a) the presence of at least one of PTM-1, PTM7, PTM-10 and PTM-11, and
(b) the absence of at least one of PTM-4 and PTM-5;

wherein VD is indicated by:
(a) the presence of at least one of PTM-5 and PTM-5, and
(b) the absence of at least one of PTM-1, PTM-7, pTM-10 and PTM-11;

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

An in vitro or ex vivo method to differentiate cognitive decline of an asymptomatic AD (CU to AD) from a neurodegenerative disease selected from the following:, MCI with a prognosis of cognitive decline of AD (MCI to AD), Alzheimer's disease (AD), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein CU is differentiated from a said neurodegenerative disease as follows (A)-(E):

(A) CU to AD is differentiated from MCI to AD:

wherein CU to AD is indicated by:
(a) the presence of at least one of PTM-4, and PTM-9, and
(b) the absence of at least one of PTM-1, PTM-3, PTM-6, PTM-7, PTM-10 and PTM-11;

wherein MCI to AD is indicated by:
(a) the presence of at least one of PTM-1, PTM-3, PTM-6, PTM-7, PTM-10 and PTM-11, and
(b) the absence of at least one of PTM-4 and PTM-9;

(B) CU to AD is differentiated from AD:

wherein CU to AD is indicated by:
(a) the presence of PTM-9,
(b) the absence of at least one of PTM-1, PTM-2, PTM-3, PTM-6, and PTM-11, and
(c) no truncation of the region denoted by amino acids 1-248 of p53 protein (SEQ ID NO:1);

wherein AD is indicated by:
(a) the presence of at least one of PTM-1, PTM-2, PTM-3, PTM-6 and PTM-11, and
(b) the absence of PTM-9, and
(c) truncation of the region denoted by amino acids 1-248 of p53 protein (SEQ ID NO:1);

(C) CU to AD is differentiated from FTD:

wherein CU to AD is indicated by:
(a) the presence of PTM-4;

wherein FTD is indicated by:
(a) the absence of PTM-4;

(D) CU to AD is differentiated from LB:

wherein CU to AD is indicated by:
(a) the presence of at least one of PTM-4 and PTM-9, and
(b) the absence of at least one of PTM-2 and PTM-6;

wherein LB is indicated by:
(a) the presence of at least one of PTM-2, and PTM-6, and
(b) the absence of at least one of PTM-4 and PTM-9;

(E) CU to AD is differentiated from VD:

wherein CU to AD is indicated by:
(a) the presence of PTM-9;

wherein VD is indicated by:
(a) the absence of PTM-9;

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

An in vitro or ex vivo method to differentiate Mild Cognitive Impairment with a prognosis of cognitive decline of AD (MCI to AD) from a neurodegenerative disease selected from the following:, cognitive decline of an asymptomatic AD (CU to AD), Alzheimer's disease (AD), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein MCI is differentiated from a said neurodegenerative disease as follows (A)-(E):

(A) MCI to AD is differentiated from CU to AD:

wherein MCI to AD is indicated by:
(a) the presence of at least one of PTM-1, PTM-3, PTM-6, PTM-7, PTM-10 and PTM-11, and
(b) the absence of at least one of PTM-4 and PTM-9;

wherein CU to AD is indicated by:
(a) the presence of at least one of PTM-4, and PTM-9, and
(b) the absence of at least one of PTM-1, PTM-3, PTM-6, PTM-7, PTM-10 and PTM-11;

(B) MCI to AD is differentiated from AD:

wherein MCI to AD is indicated by:
(a) the presence of at least one of PTM-7 and PTM-10,
(b) the absence of at least one of PTM-2, and PTM-4, and
(c) no truncation of the region denoted by amino acids 1-248 of p53 protein (SEQ ID NO:1);

wherein AD is indicated by:
(a) the presence of at least one of PTM-2 and PTM-4,
(b) the absence of at least one of PTM-7 and PTM-10, and
(c) truncation of the region denoted by amino acids 1-248 of p53 protein (SEQ ID NO:1);

(C) MCI to AD is differentiated from FTD:

wherein MCI to AD is indicated by:
(a) the presence of at least one of PTM-1, PTM-3, PTM-6, PTM-7, PTM-10 and PTM-11, and
(b) the absence of PTM-9;

wherein FTD is indicated by:
(a) the presence of PTM-9, and
(b) the absence of at least one of PTM-1, PTM-3, PTM-6, PTM-7, PTM-10 and PTM-11;

(D) MCI to AD is differentiated from LB:

wherein MCI to AD is indicated by:
(a) the presence of at least one of PTM-1, PTM-3, PTM-7, PTM-10 and PTM-11, and
(b) the absence of PTM-2;

wherein LB is indicated by:
(a) the presence of PTM-2, and
(b) the absence of PTM-1, PTM-3, PTM-7, PTM-10 and PTM-11;

(E) MCI to AD is differentiated from VD:

wherein MCI to AD is indicated by:
(a) the presence of at least one of PTM-1, PTM-3, PTM-6, PTM-7, PTM-10 and PTM-11, and
(b) the absence of PTM-4;

wherein VD is indicated by:

(a) the presence of PTM-4, and

(b) the absence of at least one of PTM-1, PTM-3, PTM-6, PTM-7, PTM-10 and PTM-11;

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

An in vitro or ex vivo method to differentiate Frontotemporal dementia (FTD) from a neurodegenerative disease selected from Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (SEQ ID NO:1), said PTMs comprising PTMs-1-12, wherein FTD is differentiated from LB and from VD as follows (A) and (B), respectively:

(A) FTD is differentiated from LB:

wherein FTD is indicated by:
(a) the presence of PTM-9, and
(b) the absence of at least one of PTM 2 and PTM-6;

wherein LB is indicated by:
(a) the presence of at least one of PTM 2 and PTM-6, and
(b) the absence of PTM-9;

(B) FTD is differentiated from VD:

wherein FTD is indicated by:
(a) the presence of PTM-9, and
(b) the absence of PTM-4;

wherein VD is indicated by:
(a) the presence of PTM-4, and
(b) the absence of PTM-9;

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

An in vitro or ex vivo method to differentiate Lewi's body dementia (LB) and Vascular dementia (VD) in a subject, the method comprising detecting in a biofluid sample of said subject the presence and/or absence of one or more post-translation modifications (PTMs) in the region of amino acids 1-371 of the p53 protein (SEQ ID NO:1), said PTMs comprising PTMs-1-12,

wherein LB and VD are differentiated by:
wherein LB is indicated by:
   (a) the presence of at least one of PTM 2 and PTM-6, and
   (b) the absence of PTM-4;

and

wherein VD is indicated by:
(a) the presence of PTM-4, and
(b) the absence of PTM-2 and PTM-6;

wherein said PTMs 1-12 comprise:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

An in vitro or ex vivo method to identify in a subject a neurodegenerative disease selected from: Mild Cognitive Impairment (MCI), Frontotemporal dementia (FTD), Lewi's body (LB) and Vascular dementia (VD), cognitively unimpaired (CU) leading to AD, and MCI leading to AD, or to identify said subject as cognitively unimpaired (CU), wherein said method comprises detecting in a biofluid sample of said subject the presence and/or absence of one or more post-translation modifications (PTMs) in the region of amino acids 1-371 of the p53 protein (U-p53), said PTMs comprising PTMs-1 - 12, wherein a CU subject or a selected neurodegenerative disease is indicated as set forth in one of (A)-(H), respectively:

(A) wherein a cognitively unimpaired (CU) subject is indicated by:
(a) the presence of PTM-7, and optionally the presence of at least one of PTMs-2 and 8,
(b) no truncation of the region denoted by amino acids 1-248 of p53 protein (SEQ ID NO:1), and
(c) the absence of at least one of PTMs-1, 3, 4, 5, and 6, and

optionally the additional absence of PTMs-9 and 10,

thereby identifying AD in said subject;

(B) wherein MCI in said subject is indicated by:
(a) the presence of at least one of PTMs-7 and 10, and optionally the additional presence of at least one of PTMs-a 1, 8 and 11,
(b) no truncation of the region denoted by amino acids 1-248 of p53 protein (SEQ ID NO:1), and
(c) the absence of at least one of PTMs-2, 3, 4, 5, and 6, and optionally the additional absence of PTM-9,

thereby identifying MCI in said subject;

(C) wherein a progression of CU to AD in said subject is indicated by:
(a) the presence of at least one of PTMs-4, 5 and 9,
(b) no truncation of the region denoted by amino acids 1-248 of p53 protein (SEQ ID NO:1), and
(c) the absence of at least one of PTMs-1, 2, 3, 6, 7, 8 and

optionally the additional absence of PTM-10 and 11,

thereby identifying the progression of CU to AD in said subject;

(D) wherein a progression of MCI to AD in said subject is indicated by:
(a) the presence of at least one of PTMs-1, 3, 5, 6, 7, 8, and optionally the additional presence of at least one of PTMs-10 and 11,
(b) no truncation of the region denoted by amino acids 1-248 of p53 protein (SEQ ID NO:1), and
(c) the absence of at least one of PTMs-2, 4 and 9,

thereby identifying the progression of MCI to AD in said subject;

(E) wherein AD in said subject is indicated by:
(a) the presence of at least one of PTMs-3, 4, 5, and 6, and optionally the additional presence of at least one of PTMs-1, 2, 8 and 11,
(b) a truncation of the region denoted by amino acids 1-248 of p53 protein (SEQ ID NO:1), and
(c) the absence of at least one of PTMs-7 and 9, and optionally the additional absence of PTMs-10,

thereby to identify AD in said subject;

(F) wherein FTD in said subject is indicated by:
(a) the presence of at least one of PTMs-5, 7, 8, and 9,
(b) no truncation of the region denoted by amino acids 1-248 of p53 protein (SEQ ID NO:1), and
(c) the absence of at least one of PTMs-1, 2, 3, 4, 6, 10 and 11,

thereby to identify FTD in said subject;

(G) wherein LB in said subject is indicated by:
(a) the presence of at least one of PTMs-2, 5, 6, and 8,
(b) no truncation of the region denoted by amino acids 1-248 of p53 protein (SEQ ID NO:1), and
(c) the absence of at least one of PTMs-1, 3, 4, 7, and 9, and optionally the additional absence of at least one of PTMs-10 and 11,

thereby to identify LB in said subject;

and

(H) wherein VD in said subject is indicated by:
(a) the presence of at least one of PTMs-4, 5, and 8,
(b) no truncation of the region denoted by amino acids 1-248 of p53 protein (SEQ ID NO:1), and
(c) the absence of at least one of PTMs-1, 2, 3, .6, 7, and 9, and optionally the additional absence of at least one of PTMs-10 and 11,

thereby to identify VD in said subject;

wherein said PTMs 1-12 of (A)-(H) are defined as:

PTM-1 at the amino acid M1,

PTM-2 at the amino acid K164,

PTM-3 at the amino acid K370,

PTM-4 at the amino acid L101,

PTM-5 at the amino acid K120,

PTM-6 at the amino acid K132,

PTM-7 at the amino acid K139,

PTM-8 at the amino acid K291,

PTM-9 at the amino acid K357,

PTM-10 at the amino acid S6,

PTM-11 at the amino acid S33, and

PTM-12 is the truncation of amino acids 1-248 of SEQ ID NO: 1.

The in vitro or ex vivo method as set forth herein, wherein said biofluid is blood, plasma, serum, saliva, urine, cerebrospinal fluid and neuronal cells, preferably said biofluid is blood, preferably said biofluid is plasma, and preferably said biofluid is cerebrospinal fluid.

The in vitro or ex vivo method as set forth herein, wherein the p53 protein is captured in a biofluid sample by performing the following steps of:

(i) providing a biofluid sample;

(ii) performing protein immunoprecipitation by an antibody that binds a p53 protein, said antibody comprising heavy chain CDRs 1, 2 and 3 (SEQ ID NOs: 11, 12 and 13, respectively) and light chain CDRs 1, 2 and 3 (SEQ ID NOs: 14, 15 and 16, respectively).;

(iii) performing protein fragmentation by trypsin.

The in vitro or ex vivo method as set forth herein1, wherein said PTMs are detected by HPLC-mass spectrometry and/or Edman degradation.

The in vitro or ex vivo method as set forth herein, wherein:

the post-translation modification PTM-1 has a group CO—CH₃ branched to the amino acid M1 of the p53 protein;

the post-translation modification PTM-2 has a group CO—CH₃ branched to the amino acid K164 of the p53 protein;

the post-translation modification PTM-3 has a group CO—CH₃ branched to the amino acid K370 of the p53 protein;

the post-translation modification PTM-4 has a ubiquitination site [GG] branched at the amino acid K101 of the p53 protein;

the post-translation modification PTM-5 has a ubiquitination site [GG] branched at the amino acid K120 of the p53 protein;

the post-translation modification PTM-6 has a ubiquitination site [GG] branched at the amino acid K132 of the p53 protein;

the post-translation modification PTM-7 has a ubiquitination site [GG] branched at the amino acid K139 of the p53 protein;

the post-translation modification PTM-8 has a ubiquitination site [GG] branched at the amino acid K291 of the p53 protein;

the post-translation modification PTM-9 has a ubiquitination site [GG] branched at the amino acid K357 of the p53 protein;

the post-translation modification PTM-10 has phosphorylation at the amino acid S6 of the p53 protein;

the post-translation modification PTM-11 has phosphorylation at the amino acid

As said above, it should be appreciated that said variability and truncation are peculiar of Alzheimer's disease, as the same are not detected in biofluid samples of patients affected by other forms of dementia. At the same time, a residual amount of U-p53 in the biofluid samples keeps its sequence length, whereon peculiar PTMs of Alzheimer's disease are detected. It follows that patients affected by Alzheimer's disease are unequivocally identified and distinguished from other dementia patients, insofar as the former show both a truncation in the U-p53 protein sequence and peculiar PTMs in the residual amount of untruncated U-p53 protein.

Preferably, in the in vitro or ex vivo method of the present invention, the presence of all PTM-2, PTM-7, PTM-8, and PTM-11 is indicative of a cognitive unimpaired subject (CU).

Preferably, in the in vitro or ex vivo method of the present invention the presence of PTM-1, and PTM-10 is indicative of MCI.

Preferably, in the in vitro or ex vivo method of the present invention the presence of at least two PTMs selected from PTM-4, PTM-5, and PTM-9 is indicative of an asymptomatic subject having the prognosis of cognitive decline of Alzheimer's dementia (AD), more preferably the presence of all PTM-4, PTM-5, and PTM-9. In this regard, it should be appreciated that the method of the invention allows the cognitive unimpaired subject (CU) to be identified and distinguished from the asymptomatic subject having the prognosis of cognitive decline of Alzheimer's dementia, although both subjects are formally asymptomatic and accordingly not distinguishable from each other through conventional cognitive tests.

Preferably, in the in vitro or ex vivo method of the present invention the presence of at least two PTMs selected from PTM-1, PTM-3, PTM-5, PTM-6, and PTM-10 is indicative of MCI with a prognosis of cognitive decline of AD, more preferably the presence of all PTM-1, PTM-3, PTM-5, PTM-6, and PTM-10.

In some embodiments, the presence of PTM 9 and the absence of truncation in the region denoted by amino acids 1-248 of wild type p53 (SEQ ID NO: 1), indicates that the patient has FTD.

Preferably, the presence of PTM 9, the absence of truncation in the region denoted by amino acids 1-248 of wild type p53 (SEQ ID NO: 1), and the absence of at least one or more of PTMs-1, 2, 3, 4, 6 and 11 indicates that the patient has FTD

Preferably, in the in vitro or ex vivo method of the present invention the presence of PTM-5, and PTM-9 is indicative of FTD.

In some embodiments, the absence of at least one of PTMs-1, 3, 4, and 11 and the absence of truncation in the region denoted by amino acids 1-248 of wild type p53 (SEQ ID NO: 1), indicates that the patient has LB.

Preferably, in vitro or ex vivo method of the present invention the presence of PTM-5, and PTM-6 is indicative of LB.

In some embodiments, the absence of one or more of PTMs 1, 2, 3, 6 and 11 and the absence of truncation in the region denoted by amino acids 1-248 of wild type p53 (SEQ ID NO: 1), indicates that the patient has VD.

Preferably, in the in vitro or ex vivo method of the present invention the presence of PTM-4, and PTM-5 is indicative of VD.

Preferably, said biofluid is blood, plasma, cerebrospinal fluid, serum, saliva, urine, neuronal cells, blood cells or other types of cells.

According to a preferred embodiment, in the step a) of the in vitro or ex vivo method of the present invention, the p53 protein is captured in a biofluid sample by performing the following sub-steps of:

(i) providing a biofluid sample;

(ii) performing protein immunoprecipitation by an antibody that binds a p53 protein;

(iii) performing protein fragmentation by trypsin;

and the step b) is performed by HPLC-mass spectrometry, Peptide Mass Fingerprint and Database Search.

In a preferred embodiment, the p53 protein in step a) is the U-p53 in a misfolded conformation.

Preferably, the antibody of sub-step (ii) is a conformationally specific antibody that binds to a p53 peptide, more preferably is a monoclonal/polyclonal antibody. In preferred embodiments, said monoclonal antibody is the antibody 2D3A8.

The amino acid sequences of the 2D3A8 antibody include the heavy chain (SEQ ID NO: 7) and light chain (SEQ ID NO: 8), heavy chain variable region (SEQ ID NO: 9) and light chain variable region (SEQ ID NO: 10), heavy chain CDRs 1, 2 and 3 (SEQ ID NOs: 11, 12 and 13, respectively) and light chain CDRs 1, 2 and 3 (SEQ ID NOs: 14, 15 and 16, respectively).

Preferably, the biological sample of step a) is subjected to protein plasma depletion by HPLC or chromatographic columns or chemical treatment, before performing step (ii).

In a preferred embodiment, in the step c) of the method of the present invention, the detected PTMs are correlated with the diagnosis/prognosis of Alzheimer's disease in a patient at different stages of the diseases or cognitive decline due to dementia.

Preferably, in the step c) the detected PTMs are correlated with the prognosis of cognitive decline of Alzheimer's disease in asymptomatic individuals and subjects suffering from MCI.

In a further aspect, the present invention also relates to a diagnostic kit to be used for the implementation of the in vitro or ex vivo method above described, the kit comprising the reagent set to perform the immunoprecipitation including an antibody, the digestion of the protein (preferably trypsin with/without Lys C), elution buffer to precipitate the protein captured by the antibody, and an injection buffer.

In further aspects, the present invention also relates to a method for detecting neurodegenerative disease or development of neurodegenerative disease in a subject by identifying the type of post-translational modifications (PTMs) in the region of amino acids 1-371 of the p53 protein (U-p53) present in a sample from said subject, the method comprising the steps of:
a. subjecting said sample to immunoprecipitation with an antibody that binds to an amino acid sequence defined by amino acids 282-297 of U-p53;
b. subjecting said immunoprecipitated sample of step (a) to protease digestion;
c. detecting the presence of post-translation modifications (PTMs) in the region of amino acids 1-371 of the p53 protein (U-p53) in said digested sample of step (b) and classifying the PTM as PTM-1, PTM-2, PTM-3, PTM-4, PTM-5, PTM-6, PTM-7, PTM-8, PTM-9, PTM-10 and PTM-11,

wherein said PTM-1 is at the amino acid M1 of said U-p53, said PTM-2 is at the amino acid K164 of said U-p53, said PTM-3 is at the amino acid K370 of said U-p53, said PTM-4 is at the amino acid L101 of said U-p53, said PTM-5 is at the amino acid K120 of said U-p53, said PTM-6 is at the amino acid K132 of said U-p53, said PTM-7 is at the amino acid K139 of said U-p53, said PTM-8 is at the amino acid K291 of said U-p53, said PTM-9 is at the amino acid K357 of said U-p53, said PTM-10 is at the amino acid S6 of said U-p53, and said PTM-11 is at the amino acid S33 of said U-p53,

wherein the presence of at least two PTMs selected from PTM-1, PTM-3, PTM-4, PTM-5, PTM-6, PTM-9, and PTM-10, and the presence of at least one PTM selected from PTM-2, PTM-7, PTM-8, and PTM-11 is indicative of neurogenerative disease or development of neurodegenerative disease,

wherein said neurodegenerative disease is Alzheimer's disease, cognitive decline to Alzheimer's disease (AD), Mild cognitive impairment (MCI), Mild cognitive impairment (MCI) with a prognosis of cognitive decline to AD, Frontotemporal dementia (FTD), and/or Lewy Body's Dementia (LB), and vascular dementia (VD).

According to the present invention, preferably in said method said PTM-1 has a group CO—CH3 branched to the amino acid M1 of the p53 protein; said PTM-2 has a group CO—CH3 branched to the amino acid K164 of the p53 protein; said PTM-3 has a group CO—CH3 branched to the amino acid K370 of the p53 protein; said PTM-4 has a ubiquitination site [GG] branched at the amino acid K101 of the p53 protein; said PTM-5 has a ubiquitination site [GG] branched 10 at the amino acid K120 of the p53 protein; said PTM-6 has a ubiquitination site [GG] branched at the amino acid K132 of the p53 protein; said PTM-7 has a ubiquitination site [GG] branched at the amino acid K139 of the p53 protein; said PTM-8 has a ubiquitination site [GG] branched at the amino acid K291 of the p53 protein; said PTM-9 has a ubiquitination site [GG] branched at the amino acid K357 of the p53 protein; said PTM-10 has phosphorylation at the amino acid S6 of the p53 protein; and said PTM-11 has phosphorylation at the amino acid S33 of the p53 protein.

Preferably in said method, said at least two PTMs detected in step (c) are selected from the group consisting of PTM-1, PTM-3, PTM-4, PTM-5, and PTM-6, said detection being indicative of Alzheimer's disease (AD) or prognosis of AD.

Preferably, in said method, presence of truncation in the region denoted by the amino acids 1-248 of SEQ ID NO: 1 and presence of at least one of PTMs-3, 4, 5 and 6 and optionally the additional presence of at least one of PTMs-1, 2, 8 and 11, and the absence of at least one of PTM-7 and PTM-9 indicate that the patient is affected by AD.

Preferably in said method, said at least two PTMs detected in step (c) are selected from the group consisting of PTM-1, and PTM-10, said detection being indicative of MCI.

Preferably, in said method, the presence of at least one of PTMs 7 and 10, and optionally the additional presence of at least one of PTMs-a 1, 8 and 11, the absence of truncation in the region denoted by amino acids 1-248 of wild type p53 (SEQ ID NO: 1), and the absence of at least one of PTMs 2-6 and optionally the additional absence of PTM-9, indicates that the patient has MCI.

Preferably in said method, said sample is from a subject who exhibits no symptoms of AD, wherein said at least two PTMs detected in step (c) are selected from the group consisting of PTM-4, PTM-5, and PTM-9, said detection being indicative of a prognosis of cognitive decline to AD.

Preferably in said method, said at least two PTMs detected in step (c) are selected from the group consisting of PTM-1, PTM-3, PTM-5, PTM-6, and PTM-10, said detection being indicative of MCI with a prognosis of cognitive decline to AD.

Preferably in said method, said at least two PTMs detected in step (c) are selected from the group consisting of PTM-5, and PTM-9, said detection being indicative of FTD.

Preferably in said method, the absence of at least one of PTMs-1, 2, 3, 4, 6, 10 and 11, and the absence of truncation in the region denoted by amino acids 1-248 of p53 (SEQ ID NO: 1), the presence of at least one of PTMs-5, 7, 8, and 9, indicates that the patient has FTD.

Preferably in said method, said at least two PTMs detected in step (c) are selected from the group consisting of PTM-5, and PTM-6, said detection being indicative of LB.

Preferably in said method, the presence of at least one of PTMs-2, 5, 6, and 8, and the absence of truncation in the region denoted by amino acids 1-248 of p53 (SEQ ID NO: 1), and the absence of at least one of PTMs-1, 3, 4, 7, and 9, and optionally the additional absence of at least one of PTMs-10 and 11, indicates that the patient has LBPreferably in said method, said at least two PTMs detected in step (c) are selected from the group consisting of PTM-4, and PTM-5, said detection being indicative of VD.

Preferably in said method, the presence of at least one of PTMs-4, 5, and 8, absence of truncation of the region denoted by amino acids 1-271 248 of p53 protein (SEQ ID NO:1), and the absence of at least one of PTMs-1, 2, 3, .6, 7, and 9, and optionally the additional absence of at least one of PTMs-10 and 11 indicate VD.

Preferably in said method, said sample is selected from the group consisting of blood, plasma, cerebrospinal fluid, serum, saliva, urine, neuronal cells.

Preferably in said method, said protease is trypsin.

Preferably in said method, said detection of step (c) is performed by one or more of HPLC-mass spectrometry, Peptide Mass Fingerprint and Database search.

Preferably in said method, said antibody is a monoclonal antibody, more preferably it is 2D3A8.

Preferably in said method, said sample is subjected to protein plasma depletion by HPLC or chromatographic columns or chemical treatment, prior to performing steps (a) to (c).

In further aspects, the present invention also relates to a kit for detecting neurodegenerative disease or development of neurodegenerative disease in a subject, the kit comprising a reagent set to perform immunoprecipitation, said reagent set comprising an anti-human p53 antibody capable of binding to an amino acid sequence defined by amino acids 282-297 of U-p53, preferably wherein said anti-human p53 antibody being a monoclonal antibody, more preferably said monoclonal antibody being 2D3A8.

It should be also understood that all the combinations of preferred aspects of the peptides of the invention, as well as of the preparation processes, kit and methods using of the same, as above reported, are to be deemed as hereby disclosed. All combinations of the preferred aspects of the PTMs of the invention, preparation processes, kit and methods disclosed above are to be understood as herein described.

Below are working examples of the present invention provided for illustrative purposes.

### Materials and Methods

### Isolation and Identification of the U-p53 Protein Sequences and of its Post-Translational Modifications

The analysis relates to the identification of the U-p53 protein sequence and of its post translational modifications when extracted from plasma of cognitive unimpaired subjects (CU), of patients affected by AD, of other forms of dementia (FTD, LB and VD) and from individuals with Mild Cognitive Decline (MCI), from MCI patients with a prognosis of cognitive decline of AD (MCI to AD) and from patients with a prognosis of cognitive decline of an asymptomatic AD (CU to AD).

Treatment

The treatment regimen for neurological diseases varies from one another. For example, galantamine, rivastigmine, and donepezil are cholinesterase inhibitors that are routinely prescribed for the treatment of AD. However, for MCI, drugs such as benzodiazepines and Anticholinergics are prescribed. Likewise, for FTD, antidepressants such as trazodone, Selective serotonin reuptake inhibitors (SSRIs)—such as citalopram (Celexa), paroxetine (Paxil) or sertraline (Zoloft) and antipsychotic medications, such as olanzapine (Zyprexa) or quetiapine (Seroquel) are commonly prescribed.

Anti-inflammatory drugs may include, for example, a steroid and/or mesalazine, a corticosteroid such as prednisolone or budesonide and wherein said immunosuppressive drug administration comprises administering a TNFa inhibitor such as infliximab, azathioprine, methotrexate and/or 6-mercaptopurine.

Anti-inflammatory drugs also may be used to treat neurodegenerative diseases recited herein: Celecoxib, diclofenac, ibuprofen, paracetamol, aspirin and naproxen. See, for example, Jack Rivers-Auty, Alison E Mather, Ruth Peters, Catherine B Lawrence, David Brough, Alzheimer's Disease Neuroimaging Initiative, Anti-inflammatories in Alzheimer's disease—potential therapy or spurious correlate?, Brain Communications, Volume 2, Issue 2, 2020, fcaa109, the contents of which are herein incorporated by reference.

Symptoms of neurodegenerative diseases set forth herein are generally known in the art and are identifiable by a medical expert in neurodegeneration, and include those identified in, for example, Levenson RW, Sturm VE, Haase CM. Emotional and behavioral symptoms in neurodegenerative disease: a model for studying the neural bases of psychopathology. Annu Rev Clin Psychol. 2014;10:581-606. doi:10.1146/annurev-clinpsy-032813-153653, the contents of which are herein incorporated by reference.

Many of neurological diseases are progressive in nature, therefore early detection to identify the specific type of neurodegenerative disease followed by early treatment is of paramount importance. Since the process of treatment and recovery is a long-term process, there is thus a need for early detection and treatment followed by regular monitoring to ensure that the specific type of neurodegenerative disease is not progressing when being treated with the selected drug. Early introduction of aggressive therapy in a given neurodegenerative disease leads to better outcome.

### Sample Preparation

### 1. Buffers

Buffer A: Tris 25 mM, Sodium Chloride (NaCl) 0.15 mM, Tween-20 50 mM; Preparation: Tris (303 mg), Sodium Chloride (NaCl; 885 mg) and Tween-20 (5.5 g) are collected. Bidistilled water is added so to reach 100 mL final volume. Note: The solution must be fresh prepared for each analytical section.

Buffer B: Glycine 0.1 M pH 2.0. Preparation: Glycine (750 mg) Glycine is treated with bidistilled water. 100 mL solution was obtained. HC10.1 M is added to obtain pH 3 value. Note: The solution must be fresh prepared for each analytical section.

Ammonium bicarbonate (NH4HCO3) 0.4 g are solubilized in 100 mL of Bidistilled Water. Note: solution pH should be checked before to proceed with the analysis. pH must be lower than 8 to obtain a reproducible digestion.

### 2. Reagent Preparation

Dithiothreitol (DTT) 180 mM in 50 mM AmBic. Procedure: DTT 0.3 g are solubilized in 0.5 mL of bidistilled water. 10 mL of 50 mM ammonium bicarbonate (NH4HClO3) are added. Solubilize the mixture by using vortex. Note: The solution must be fresh prepared for each analytical section.

Iodoacetamide (IAA) 400 mM in 50 mM AmBic. Procedure: Iodoacetamide (IAA) 0.7 g are solubilized in 10 mL of 50 mM ammonium bicarbonate (NH4HCO3) solution. Solubilize the mixture by using vortex. Note: The solution must be fresh prepared for each analytical section.

25 ng/μL Trypsin solution. Procedure: 20 μg of trypsin are solubilized 800 μL of 50 mM NH4HCO3. Solubilize the mixture by using vortex. Note: The solution must be fresh prepared for each analytical section.

### 3. Bead-Antibody Binding

Protein magnetic bead L 50 μL (0.5 mg) are collected in a Vial;
150 μL Buffer A are added. Vortex is applied;
Magnetic surface is used to discard the surnatant.
Buffer A 1 mL is added. Vortex is applied for 1 minute;
Magnetic surface is used to discard the surnatant;
Antibody solution (200 μL, 0.05 μg/ μL corresponding to 10 μg) is added to ProteinL magnetic bead;
The solution is mixed for 2 hours;
Magnetic surface is used to discard the surnatant;
Buffer A 500 μL is added;
Magnetic surface is used to discard the surnatant;
Wash and discard the surnatant again;
Buffer A 1 mL is added.
The solution is stored at room temperature.

### 4. Plasma Chemical Contaminants Depletion and Immune Precipitation

Samples extracted from the different categories of patients are thawed at room temperature under laminar flow cabinet for 30 min.
The sample is spiked in 25 μL aliquots. They are separately processed.
The remaining material is stored at -20° C. for retesting purpose.
5 μL of CH3CN are added to 25 μL of plasma.
The acetonitrile spike is repeated every 1 minute since to reach a mixture volume of 50 μL. Apply vortex for 5 minutes until when white deposit is observed.
The sample centrifugation takes place at 13000 g for 10 minutes. 40 μL of surnatant is added to the bead-antibody complex. Vortex is weakly applied.
The mixture is incubated at room temperature for 1 hour and then at 4° overnight.
A magnetic surface is used to remove the surnatant.
Buffer A 500 μL are added and the mixture was vortexed.
A magnetic plane is used to remove the surnatant.
Buffer B 45 μL are added to the pellet. After mixing, to incubate for 10 minutes at room temperature.
A magnetic surface is used to collect the eluate (40 μL) that is is enzymatically digested.

### 5. Enzymatic Digestion of the Immunocaptured p53 Protein

2.15 μl of Dithiothreitol (DTT) 180 mM are added to 40 μL of the eluate.
The mixture is incubated for 15 min at 50° C. and at room temperature for 30 minutes;
2.15 μL of Iodoacetamide (IAA) 400 mM are added 42.15 μL of the mixture.
The obtained mixture is incubated for 15 minutes at room temperature.
2.15 μL of AmBic 50 mM are added 44.30 μL of the obtained mixture.
1 μL of trypsin (25 ng/μL) containing Lys-c (50 ng/μL) and AmBic 50 mM is added to 46.45 μL of the obtained mixture.
Incubation takes place at 37° C. for 3.5 hours followed by 57° C. for 30 minutes.
1 μL of Formic Acid (HCOOH) is added to 47.45 μL of the obtained mixture to stop the enzymatic digestion. pH value is checked and it has to be in the range 1-4. If it is higher than 4 progressive volume (1 μL) of Formic Acid is added to obtain a pH value between 1 and 4. 10 μL of the obtained sample are analysed.

### 6. Detection of PTMs by LC-SACI-MS

HPLC Ultimate 3000 (Thermofisher, USA) with a Phenomenex Kinetex PFP 50×4.1 mm 2.6 μm are used to perform the chromatographic analysis. Binary gradient is used: Phase A (H₂O+0.2% Formic Acid (HCOOH)) and Phase C acetonitrile (CH₃CN). The gradient is reported in the table below. 10 μL of sample are injected.

LTQ Orbitrap XL is used for the data acquisition. SACI ionization source is employed. The potential surface is 47 V, Gas nebulizer pressure is 75 Psi and dry gas flow is 1.0 L/min. 350° C. of nebulizer temperature was employed together with 320° C. of dry gas one. SACI peptide adduct profile mode is employed for data acquisition (Cristoni et al. Rapid Commun Mass Spectrom. 2003;17(17):1973-81.).

TABLE 1
Chromatographic gradient.
Gradient

| Time (minute) | % C | Flow (mL/min) |
|---|---|---|
| 0 | 2% | 0.250 |
| 2.5 | 2% | 0.250 |
| 3 | 80% | 0.250 |
| 7 | 80% | 0.250 |
| 8 | 2% | 0.250 |

### 7. Data Extraction and Protein Characterization

Protein sequence and PTM data is obtained using the SANIST-prot tool operating in bottom up conditions.

Correlation between p53 Sequence Peptide and AD Diagnosis.

The plasma samples of 7 patients affected by AD, 5 cognitive unimpaired (CU), 2 patients affected by MCI, 6 frontal dementia (FD), 1 patient with vascular dementia (VD) and 1 patient with Lewy Body dementia (LB) and 6 patients with MCI to AD and 6 patients CU to AD have been treated with the experimental protocol based on protein L to isolate protein p53 disclosed above. Said protein has been exposed to double enzymatic digestion (Lys-C+trypsin) in order to maximize the peptide recovery.

| Sample ID* | N | Diagnosis |
|---|---|---|
| 1-AD; 2-AD; 3-AD; 4-AD; 5-AD; 4-S; 7-S | 7 | AD |
| 1-C; 2-C; 3-C; 4-C; 5-C | 5 | CU |
| 9D-MCI; 10D-MCI | 2 | MCI |
| 1-S; 2-S; 5-S; 8-S; 10-S; 13-S | 6 | CU to AD |
| 3-S; 6-S; 9-S; 11-S; 12-S; 14-S | 6 | MCI to AD |
| 1D-FD; 2D-FD; 3D-FD; 4D-FD; 5D-FD; 6D-FD | 6 | FTD |
| 8D-FD V | 1 | VD |
| 7D-FD | 1 | LB |

*Sample ID is a mere code exclusively used to label the samples and, as such, have no correlation to the subsequent diagnosis of corresponding patients

### Results Obtained

1. U-p53 Protein Immunocaptured from Subjects AD

The p53 protein extracted from AD individuals results truncated in the region of amino acid 1-248 with respect to the wt p53 protein (SEQ ID NO: 1) Database: UniProtKB, Protein ID: P04637, amino acids: 1 - 393). Different mistakes of enzymatic digestion have been reported that lead to the presence of variable regions, inter-subjects, between the residuals 249-371 of the truncated protein.

In Table 2 are reported the p53 linear sequences identified in AD patients and the respective molecular weight (MW).

TABLE 2

| | | | MW |
|---|---|---|---|
| Sample ID | Sequence | SEQ ID | (Da) |
| 1-AD | EVRVCACPGRDRRTEEENLR | SEQ ID NO: 2 | 11425 |
| | KKGEPHHELPPGSTKRALPN | | |
| | NTSSSPQPKKKPLDGEYFTL | | |
| | QIRGRERFEMFRELNEALEL | | |
| | KDAQAGKEPGGSRAHSSHLKS | | |
| 2-AD | RPILTIITLEDSSGNLLGRN | SEQ ID NO: 3 | 13823 |
| | SFEVRVCACPGRDRRTEEEN | | |
| | LRKKGEPHHELPPGSTKRAL | | |
| | PNNTSSSPQPKKKPLDGEYF | | |
| | TLQIRGRERFEMFRELNEAL | | |
| | ELKDAQAGKEPGGSRAHSSH | | |
| | LKS | | |
| 3-AD | SGNLLGRNSFEVRVCACPGR | SEQ ID NO: 4 | 12471 |
| | DRRTEEENLRKKGEPHHELP | | |
| | PGSTKRALPNNTSSSPQPKK | | |
| | KPLDGEYFTLQIRGRERFEM | | |
| | FRELNEALELKDAQAGKEPG | | |
| | GSRAHSSHLKS | | |
| 4-AD | TLEDSSGNLLGRNSFEVRVC | SEQ ID NO: 5 | 13016 |
| | ACPGRDRRTEEENLRKKGEP | | |
| | HHELPPGSTKRALPNNTSSS | | |
| | PQPKKKPLDGEYFTLQIRGR | | |
| | ERFEMFRELNEALELKDAQA | | |
| | GKEPGGSRAHSSHLKS | | |
| 5-AD | EVRVCACPGRDRRTEEENLR | SEQ ID NO: 2 | 11425 |
| | KKGEPHHELPPGSTKRALPN | | |
| | NTSSSPQPKKKPLDGEYFTL | | |
| | QIRGRERFEMFRELNEALEL | | |
| | KDAQAGKEPGGSRAHSSHLKS | | |
| 4-S | EVRVCACPGRDRRTEEENLR | SEQ ID NO: 2 | 11425 |
| | KKGEPHHELPPGSTKRALPN | | |
| | NTSSSPQPKKKPLDGEYFTL | | |
| | QIRGRERFEMFRELNEALEL | | |
| | KDAQAGKEPGGSRAHSSHLKS | | |
| 7-S | RPILTIITLEDSSGNLLGRN | SEQ ID NO: 3 | 13823 |
| | SFEVRVCACPGRDRRTEEEN | | |
| | LRKKGEPHHELPPGSTKRAL | | |
| | PNNTSSSPQPKKKPLDGEYF | | |
| | TLQIRGRERFEMFRELNEAL | | |
| | ELKDAQAGKEPGGSRAHSSH | | |
| | LKS | | |
| MW- | | | 12432 |
| average | | | |

2. U-p53 Immunocaptured from Cognitive Unimpaired (CU) and Cognitive Unimpaired to AD Patients.

The linear sequence of p53 extracted from 5 Cognitive unimpaired patients and 6 Cognitive unimpaired later declined to AD correspond to the entire sequence with 1-371 amino acids (SEQ ID N. 6), with a molecular weight of 41134 Da. No residuals corresponding to the region 372-391 have been identified. Table 3 reports the linear sequences obtained from the Cognitive unimpaired and Cognitive unimpaired to AD patients.

TABLE 3

| Sample ID | Sequence | SEQ ID | MW (Da) |
|---|---|---|---|
| 1-C; | MEEPQSDPSVEPPLSQETFSDLWKLLPENNVL | SEQ ID NO: 6 | 41139 |
| 2-C; | SPLPSQAMDDLMLSPDDIEQWFTEDPGPDEAP | | |
| 3-C; | RMPEAAPPVAPAPAAPTPAAPAPAPSWPLSSS | | |
| 4-C; | VPSQKTYQGSYGFRLGFLHSGTAKSVTCTYSP | | |
| 5-C; | ALNKMFCQLAKTCPVQLWVDSTPPPGTRVRA | | |
| 1-S; | MAIYKQSQHMTEVVRRCPHHERCSDSDGLAP | | |
| 2-S; | PQHLIRVEGNLRVEYLDDRNTFRHSVVVPYEP | | |
| 5-S; | PEVGSDCTTIHYNYMCNSSCMGGMNRRPILTI | | |
| 8-S; | ITLEDSSGNLLGRNSFEVRVCACPGRDRRTEE | | |
| 10-S; | ENLRKKGEPHHELPPGSTKRALPNNTSSSPQP | | |
| 13-S | KKKPLDGEYFTLQIRGRERFEMFRELNEALEL | | |
| | KDAQAGKEPGGSRAHSSHLKS | | |
| MW-average | | | 41139 |

3. U-p53 Protein Immunocaptured from Subjects Affected by Fronto-Temporal Dementia, Lewy Body's Dementia, Vascular Dementia, Mild Cognitive Decline (MCI) and MCI to AD

The results obtained from 16 subjects (6 with frontotemporal dementia, 1 with vascular dementia, 1 with Lewy Body's dementia, 2 MCI subjects and 6 MCI who developed AD) report the presence of the whole protein of 1-371 residuals. Table 4 reports the linear protein sequences of the tested subjects.

TABLE 4

| Sample ID | Sequence | SEQ ID | MW (Da) |
|---|---|---|---|
| 1D-FD; | MEEPQSDPSVEPPLSQETFSDLWKLLPEN | SEQ ID NO: 6 | 41139 |
| 2D-FD; | NVLSPLPSQAMDDLMLSPDDIEQWFTED | | |
| 3D-FD; | PGPDEAPRMPEAAPPVAPAPAAPTPAAPA | | |
| 4D-FD; | PAPSWPLSSSVPSQKTYQGSYGFRLGFLH | | |
| 5D-FD; | SGTAKSVTCTYSPALNKMFCQLAKTCPV | | |
| 6D-FD; | QLWVDSTPPPGTRVRAMAIYKQSQHMTE | | |
| 7D-FD; | VVRRCPHHERCSDSDGLAPPQHLIRVEGN | | |
| 8D-FD V; | LRVEYLDDRNTFRHSVVVPYEPPEVGSD | | |
| 9D-MCI; | CTTIHYNYMCNSSCMGGMNRRPILTIITL | | |
| 10D-MCI; | EDSSGNLLGRNSFEVRVCACPGRDRRTEE | | |
| 3-S; | ENLRKKGEPHHELPPGSTKRALPNNTSSS | | |
| 6-S; | PQPKKKPLDGEYFTLQIRGRERFEMFREL | | |
| 9.S; | NEALELKDAQAGKEPGGSRAHS SHLKS | | |
| 11-S | | | |
| 12-S | | | |
| 14-S | | | |
| MW-average | | | 41139 |

4. Description of the PTMs Observed from the Immunocaptured Protein

The extracted and sequenced p53 protein from the different clinical groups in addition to a different linear sequence, corresponding accordingly to different molecular weight, also showed post-transductional modifications (PTMs), mainly characterized by ubiquitination, acetylation and phosphorylation on specific amino acid residues. The samples belonging to the same clinical group also showed a highly homogeneity in the PTMs, which in combination with the same protein sequence represent an element characterizing the clinical group to which they belong.

In FIGS. 1-8 the ubiquitination sites observed are reported.

4.1. AD subjects

Under-expressed peptide sequences belonging to the amino acid region 1-248 were detected in AD patients. Given their low abundance, they could derive from whole sequence of p53 proteins that are believed to be weakly interacting with the antibody. The protein sequence has several ubiquitination sites indicated with the notation “ ” in FIG. 1.

### 4.2. Cognitive Unimpaired (CU) Subjects

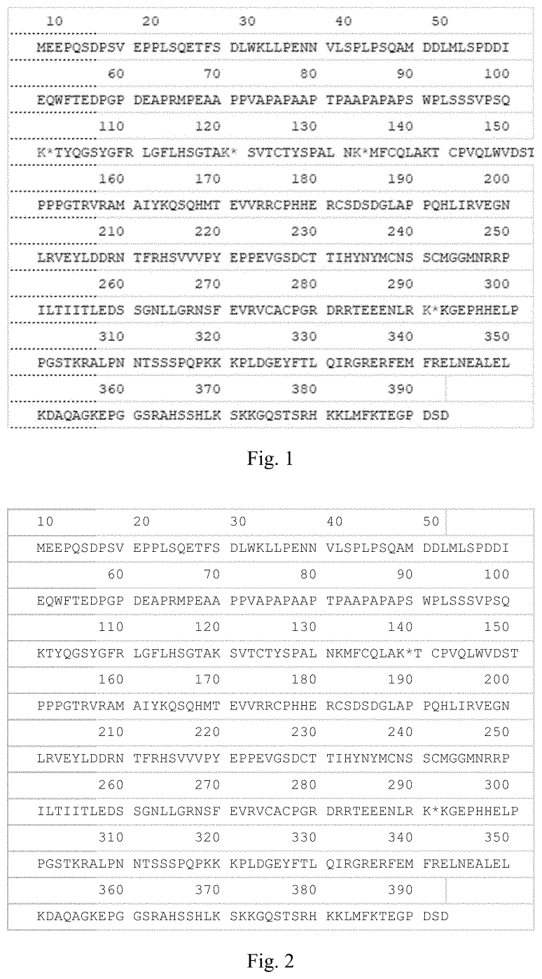

The ubiquitination sites detected in cognitive unimpaired samples are reported in FIG. 2.

### 4.3. Subjects Affected by Fronto-Temporal Dementia (FTD)

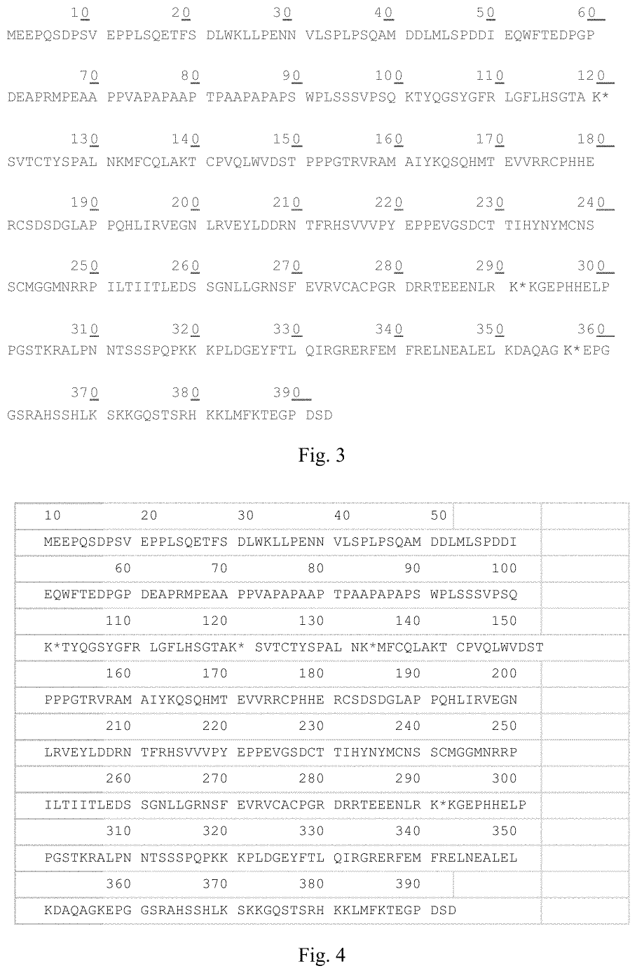

The ubiquitination sites detected in FTD samples are reported in FIG. 3.

### 4.4. Subjects Affected by Lewy Body's Dementia (LB)

The ubiquitination sites detected in LB samples are reported in FIG. 4.

### 4.5. Subjects Affected by Vascular Dementia (VD)

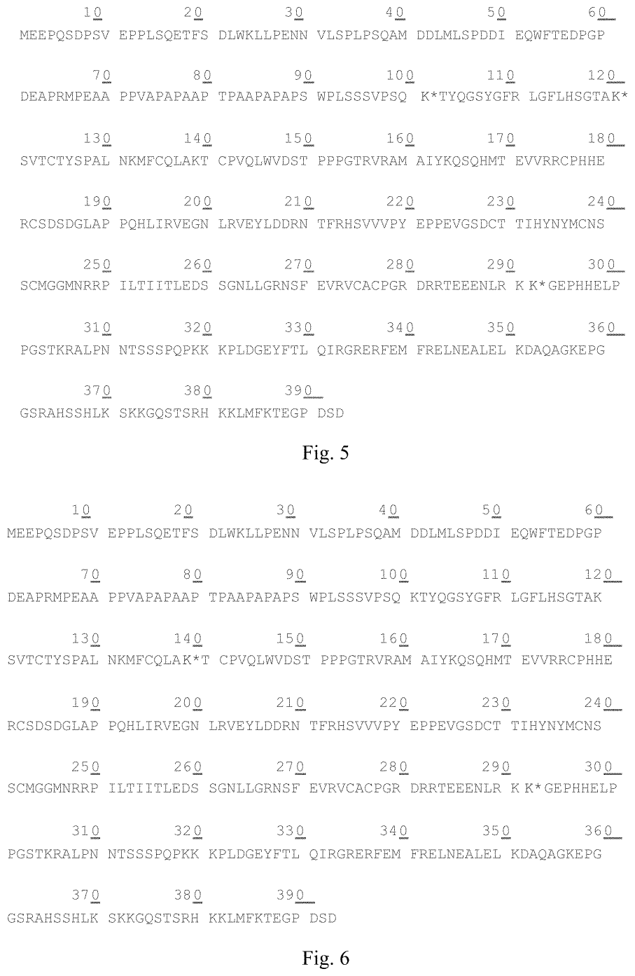

The ubiquitination sites detected in VD samples are reported in FIG. 5.

### 4.6. Subjects Affected by MCI

The ubiquitination sites detected in MCI samples are reported in FIG. 6.

### 4.7. Samples of Cognitive Unimpaired Subjects (CU) who Developed AD

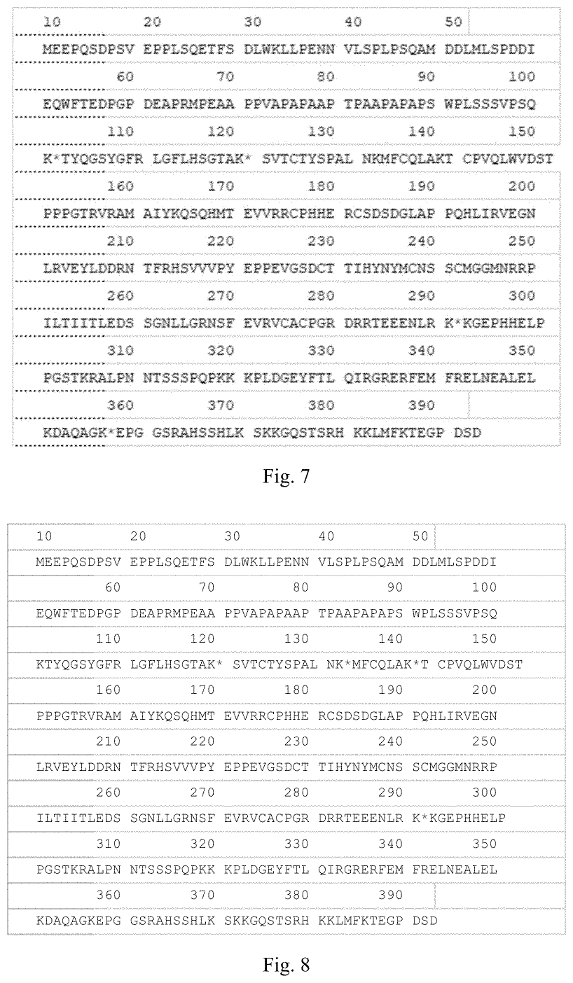

The protein ubiquitination sites detected in the samples of cognitively healthy subjects who developed AD over a period of 18-72 months are shown in FIG. 7.

### 4.8. Samples of MCI Subjects who Developed AD

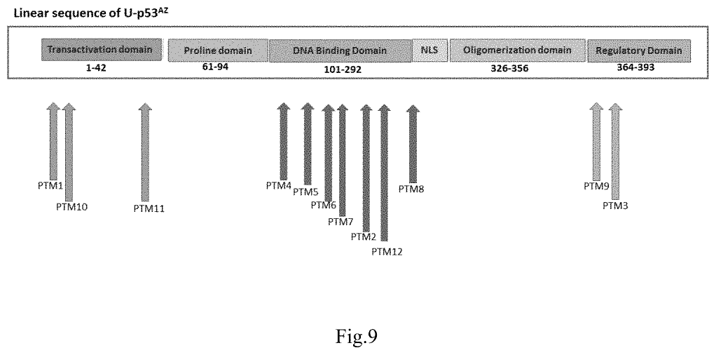

The ubiquitination sites detected in MCI subjects who developed AD are reported in FIG. 8.

From the data obtained we can observe that there were cumulatively 11 PTMs spanning the full sequence of the protein. Peptides spanning the protein up 371 residues were detected in all samples, however the peptides belonging to the region 1-248 residues AD patients seemed to be cut from the protein not as consequence of enzymatic digestion due to the analytical protocol but due to biological process of full p-53 protein. Amino acids in the region from 372 to the end of the p-53 protein was missing in all samples belonging to different clinical groups.

The PTMs observed in the different patients are disclosed in Table 5 (Y=detected; N=not detected)

TABLE 5

| | AC- | AC- | AC- | U- | U- | U- | U- | U- | U- | Phospho- | Phospho- | |
|---|---|---|---|---|---|---|---|---|---|---|---|---|
| | M1 | K164 | K370 | K101 | K120 | K132 | K139 | K291 | K357 | S6 | S33 | Truncation |
| CU | N | Y | N | N | N | N | Y | Y | N | N | Y | N |
| MCI | Y | N | N | N | N | N | Y | Y | N | Y | Y | N |
| CU to AD | N | N | N | Y | Y | N | N | Y | Y | N | N | N |
| MCI to AD | Y | N | Y | N | Y | Y | Y | Y | N | Y | Y | N |
| AD | Y | Y | Y | Y | Y | Y | N | Y | N | N | Y | Y |
| FTD | N | N | N | N | Y | N | N | Y | Y | N | N | N |
| LB | N | Y | N | Y | Y | Y | N | Y | N | N | N | N |
| VD | N | N | N | Y | Y | N | N | Y | N | N | N | N |

The information contained in Table 5 is set forth in alternative comparative formats in the following Tables:

TABLE 6
CU + MCI

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU | N | Y | N | N | N | N | Y | Y | N | N | Y | N |
| MCI | Y | N | N | N | N | N | Y | Y | N | Y | Y | N |

TABLE 7
CU + CU to AD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU | N | Y | N | N | N | N | Y | Y | N | N | Y | N |
| CU to AD | N | N | N | Y | Y | N | N | Y | Y | N | N | N |

TABLE 8
CU + MCI to AD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU | N | Y | N | N | N | N | Y | Y | N | N | Y | N |
| MCI to AD | Y | N | Y | N | Y | Y | Y | Y | N | Y | Y | N |

TABLE 9
CU + AD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU | N | Y | N | N | N | N | Y | Y | N | N | Y | N |
| AD | Y | Y | Y | Y | Y | Y | N | Y | N | N | Y | Y |

TABLE 10
CU + FTD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU | N | Y | N | N | N | N | Y | Y | N | N | Y | N |
| FTD | N | N | N | N | Y | N | N | Y | Y | N | N | N |

TABLE 11
CU + LB

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU | N | Y | N | N | N | N | Y | Y | N | N | Y | N |
| LB | N | Y | N | N | Y | Y | N | Y | N | N | N | N |

TABLE 12
CU + VD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU | N | Y | N | N | N | N | Y | Y | N | N | Y | N |
| VD | N | N | N | Y | Y | N | N | Y | N | N | N | N |

TABLE 13
CU to AD + MCI to AD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU to AD | N | N | N | Y | Y | N | N | Y | Y | N | N | N |
| MCI to AD | Y | N | Y | N | Y | Y | Y | Y | N | Y | Y | N |

TABLE 14
CU to AD + AD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU to AD | N | N | N | Y | Y | N | N | Y | Y | N | N | N |
| AD | Y | Y | Y | Y | Y | Y | N | Y | N | N | Y | Y |

TABLE 15
CU to AD + FTD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU to AD | N | N | N | Y | Y | N | N | Y | Y | N | N | N |
| FTD | N | N | N | N | Y | N | N | Y | Y | N | N | N |

TABLE 16
CU to AD + LB

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU to AD | N | N | N | Y | Y | N | N | Y | Y | N | N | N |
| LB | N | Y | N | N | Y | Y | N | Y | N | N | N | N |

TABLE 17
CU to AD + VD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| CU to AD | N | N | N | Y | Y | N | N | Y | Y | N | N | N |
| VD | N | N | N | Y | Y | N | N | Y | N | N | N | N |

TABLE 18
MCI to AD + AD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| MCI to AD | Y | N | Y | N | Y | Y | Y | Y | N | Y | Y | N |
| AD | Y | Y | Y | Y | Y | Y | N | Y | N | N | Y | Y |

TABLE 19
MCI to AD + FTD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| MCI to AD | Y | N | Y | N | Y | Y | Y | Y | N | Y | Y | N |
| FTD | N | N | N | N | Y | N | N | Y | Y | N | N | N |

TABLE 20
MCI to AD + LB

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| MCI to AD | Y | N | Y | N | Y | Y | Y | Y | N | Y | Y | N |
| LB | N | Y | N | N | Y | Y | N | Y | N | N | N | N |

TABLE 21
MCI to AD + VD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| MCI to AD | Y | N | Y | N | Y | Y | Y | Y | N | Y | Y | N |
| VD | N | N | N | Y | Y | N | N | Y | N | N | N | N |

TABLE 22
AD + FTD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| AD | Y | Y | Y | Y | Y | Y | N | Y | N | N | Y | Y |
| FTD | N | N | N | N | Y | N | N | Y | Y | N | N | N |

TABLE 23
AD + LB

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| AD | Y | Y | Y | Y | Y | Y | N | Y | N | N | Y | Y |
| LB | N | Y | N | N | Y | Y | N | Y | N | N | N | N |

TABLE 24
AD + VD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| AD | Y | Y | Y | Y | Y | Y | N | Y | N | N | Y | Y |
| VD | N | N | N | Y | Y | N | N | Y | N | N | N | N |

TABLE 25
FTD + LB

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| FTD | N | N | N | N | Y | N | N | Y | Y | N | N | N |
| LB | N | Y | N | N | Y | Y | N | Y | N | N | N | N |

TABLE 26
FTD + VD

| | 1 | | 3 | | | | 7 | | | 10 | 11 | |
|---|---|---|---|---|---|---|---|---|---|---|---|---|
| | AC | 2 | AC | 4 | 5 | 6 | U- | 8 | 9 | A6 + | S33 + | 12 |
| | M1 | AC | K370 | U101 | U120 | U132 | 139 | U291 | U357 | Phospho | Phospho | Truncated |
| FTD | N | N | N | N | Y | N | N | Y | Y | N | N | N |
| VD | N | N | N | Y | Y | N | N | Y | N | N | N | N |

TABLE 27
LB + VD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| LB | N | Y | N | N | Y | Y | N | Y | N | N | N | N |
| VD | N | N | N | Y | Y | N | N | Y | N | N | N | N |

TABLE 28
CU

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| CU | N | Y | N | N | N | N | Y | Y | N | N | Y | N |

TABLE 29
MCI

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI | Y | N | N | N | N | N | Y | Y | N | Y | Y | N |

TABLE 30
CU to AD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| CU to AD | N | N | N | Y | Y | N | N | Y | Y | N | N | N |

TABLE 31
MCI to AD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI to AD | Y | N | Y | N | Y | Y | Y | Y | N | Y | Y | N |

TABLE 32
AD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| AD | Y | Y | Y | Y | Y | Y | N | Y | N | N | Y | Y |

TABLE 33
FTD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| FTD | N | N | N | N | Y | N | N | Y | Y | N | N | N |

TABLE 34
LB

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| LB | N | Y | N | N | Y | Y | N | Y | N | N | N | N |

TABLE 35
VD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| VD | N | N | N | Y | Y | N | N | Y | N | N | N | N |

TABLE 36
MC + CU to AD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI | Y | N | N | N | N | N | Y | Y | N | Y | Y | N |
| CU to AD | N | N | N | Y | Y | N | N | Y | Y | N | N | N |

TABLE 37
MCI + MCI to AD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI | Y | N | N | N | N | N | Y | Y | N | Y | Y | N |
| MCI to AD | Y | N | Y | N | Y | Y | Y | Y | N | Y | Y | N |

TABLE 38
MC + AD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI | Y | N | N | N | N | N | Y | Y | N | Y | Y | N |
| AD | Y | Y | Y | Y | Y | Y | N | Y | N | N | Y | Y |

TABLE 39
MCI + FTD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI | Y | N | N | N | N | N | Y | Y | N | Y | Y | N |
| FTD | N | N | N | N | Y | N | N | Y | Y | N | N | N |

TABLE 40
MCI + LB

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI | Y | N | N | N | N | N | Y | Y | N | Y | Y | N |
| LB | N | Y | N | N | Y | Y | N | Y | N | N | N | N |

TABLE 41
MCI + VD

| | 1 | 2 | 3 | 4 | 5 | 6 | 7 | 8 | 9 | 10 | 11 | 12 |
|---|---|---|---|---|---|---|---|---|---|---|---|---|
| | AC M1 | AC | AC K370 | U101 | U120 | U132 | U-139 | U291 | U357 | A6 + Phospho | S33 + Phospho | Truncated |
| MCI | Y | N | N | N | N | N | Y | Y | N | Y | Y | N |
| VD | N | N | N | Y | Y | N | N | Y | N | N | N | N |

### REFERENCES

1. Stanga, S. et al., 2010. Unfolded p53 in the pathogenesis of Alzheimer's disease: Is HIPK2 the link? Aging, 2(9), pp.545-554.
2. Lanni, C. et al., 2007. Unfolded p53: A potential biomarker for Alzheimer's disease. In Journal of Alzheimer's Disease. pp. 93-99.
3. Uberti, D. et al., 2008. Conformationally altered p53: a putative peripheral marker for Alzheimer's disease. Neuro-degenerative diseases, 5(3-4), pp.209-11.
4. Lanni, C. et al., 2008. Conformationally altered p53: a novel Alzheimer's disease marker? Molecular psychiatry, 13(6), pp.641-7.
5. Lanni, C., Racchi, M., et al., 2010. Unfolded p53 in blood as a predictive signature signature of the transition from mild cognitive impairment to Alzheimer's disease. Journal of Alzheimer's disease: JAD, 20(1), pp.97-104.
6. Buizza, L. et al., 2012. Conformational altered p53 as an early marker of oxidative stress in Alzheimer's disease. PloS one, 7(1), p.e29789
7. Arce-Varas N, et al. Comparison of extracellular and intracellular blood compartments highlights redox alterations in Alzheimer's and Mild Cognitive Impairment patients. Current Alzheimer Research 2017; 14(1): 112-122.
8. Uberti, D. et al., 2006. Identification of a mutant-like conformation of p53 in fibroblasts from sporadic Alzheimer's disease patients. Neurobiology of aging, 27(9), pp.1193-201.
9. Lanni, C., Nardinocchi, L., et al., 2010. Homeodomain interacting protein kinase 2: a target for Alzheimer's beta amyloid leading to misfolded p53 and inappropriate cell survival. PloS one, 5(4), p.e10171.
10. Lanni, C. et al., 2008. Pharmacogenetics and Pharmagenomics, Trends in Normal and Pathological Aging Studies: Focus on p53. Current Pharmaceutical Design, 14(26), pp.2665-2671.
11. Peptide Mass Fingerprint (PMF; Cristoni S. et al Expert Rev Proteomics. 2004 Dec; 1(4):469-83)

```
SEQUENCE LISTING
SEQ ID NO: 1
Met Glu Glu Pro Gln Ser Asp Pro Ser Val Glu Pro Pro Leu Ser Gln
1 5 10 15
Glu Thr Phe Ser Asp Leu Trp Lys Leu Leu Pro Glu Asn Asn Val Leu
              20 25 30
Ser Pro Leu Pro Ser Gln Ala Met Asp Asp Leu Met Leu Ser Pro Asp
          35 40 45
Asp Ile Glu Gln Trp Phe Thr Glu Asp Pro Gly Pro Asp Glu Ala Pro
      50 55 60
Arg Met Pro Glu Ala Ala Pro Pro Val Ala Pro Ala Pro Ala Ala Pro
65 70 75 80
Thr Pro Ala Ala Pro Ala Pro Ala Pro Ser Trp Pro Leu Ser Ser Ser
                  85 90 95
Val Pro Ser Gln Lys Thr Tyr Gln Gly Ser Tyr Gly Phe Arg Leu Gly
              100 105 110
Phe Leu His Ser Gly Thr Ala Lys Ser Val Thr Cys Thr Tyr Ser Pro
        115 120 125
Ala Leu Asn Lys Met Phe Cys Gln Leu Ala Lys Thr Cys Pro Val Gln
    130 135 140
Leu Trp Val Asp Ser Thr Pro Pro Pro Gly Thr Arg Val Arg Ala Ala
145 150 155 160
Ile Tyr Lys Gln Ser Gln His Met Thr Glu Val Val Arg Arg Cys Pro
                    165 170 175
His His Glu Arg Cys Ser Asp Ser Asp Gly Leu Ala Pro Pro Gln His
                180 185 190
Leu Ile Arg Val Glu Gly Asn Leu Arg Val Glu Tyr Leu Asp Asp Arg
          195 200 205
Asn Thr Phe Arg His Ser Val Val Val Pro Tyr Glu Pro Pro Glu Val
    210 215 220
Gly Ser Asp Cys Thr Thr Ile His Tyr Asn Tyr Met Cys Asn Ser Ser
225 230 235 240
Cys Met Gly Gly Met Asn Arg Arg Pro Ile Leu Thr Ile Ile Thr Leu
                  245 250 255
Glu Asp Ser Ser Gly Asn Leu Leu Gly Arg Asn Ser Phe Glu Val Arg
            260 265 270
Val Cys Ala Cys Pro Gly Arg Asp Arg Arg Thr Glu Glu Glu Asn Leu
        275 280 285
Arg Lys Lys Gly Glu Pro His His Glu Leu Pro Pro Gly Ser Thr Lys
    290 295 300
Arg Ala Leu Pro Asn Asn Thr Ser Ser Ser Pro Gln Pro Lys Lys Lys
305 310 315 320
Pro Leu Asp Gly Glu Tyr Phe Thr Leu Gln Ile Arg Gly Arg Glu Arg
                  325 330 335
Phe Glu Met Phe Arg Glu Leu Asn Glu Ala Leu Glu Leu Lys Asp Ala
              340 345 350
Gln Ala Gly Lys Glu Pro Gly Gly Ser Arg Ala His Ser Ser His Leu
          355 360 365
Lys Ser Lys Lys Gly Gln Ser Thr Ser Arg His Lys Lys Leu Met Phe
    370 375 380
Lys Thr Glu Gly Pro Asp Ser Asp
385 390
SEQ ID NO: 2
Glu Val Arg Val Cys Ala Cys Pro Gly Arg Asp Arg Arg Thr Glu Glu
1 5 10 15
Glu Asn Leu Arg Lys Lys Gly Glu Pro His His Glu Leu Pro Pro Gly
              20 25 30
Ser Thr Lys Arg Ala Leu Pro Asn Asn Thr Ser Ser Ser Pro Gln Pro
        35 40 45
Lys Lys Lys Pro Leu Asp Gly Glu Tyr Phe Thr Leu Gln Ile Arg Gly
    50 55 60
Arg Glu Arg Phe Glu Met Phe Arg Glu Leu Asn Glu Ala Leu Glu Leu
65 70 75 80
Lys Asp Ala Gln Ala Gly Lys Glu Pro Gly Gly Ser Arg Ala His Ser
                  85 90 95
Ser His Leu Lys Ser
                100
SEQ ID NO: 3
Arg Pro Ile Leu Thr Ile Ile Thr Leu Glu Asp Ser Ser Gly Asn Leu
1 5 10 15
Leu Gly Arg Asn Ser Phe Glu Val Arg Val Cys Ala Cys Pro Gly Arg
              20 25 30
Asp Arg Arg Thr Glu Glu Glu Asn Leu Arg Lys Lys Gly Glu Pro His
        35 40 45
His Glu Leu Pro Pro Gly Ser Thr Lys Arg Ala Leu Pro Asn Asn Thr
    50 55 60
Ser Ser Ser Pro Gln Pro Lys Lys Lys Pro Leu Asp Gly Glu Tyr Phe
65 70 75 80
Thr Leu Gln Ile Arg Gly Arg Glu Arg Phe Glu Met Phe Arg Glu Leu
                  85 90 95
Asn Glu Ala Leu Glu Leu Lys Asp Ala Gln Ala Gly Lys Glu Pro Gly
              100 105 110
Gly Ser Arg Ala His Ser Ser His Leu Lys Ser
        115 120
SEQ ID NO: 4
Ser Gly Asn Leu Leu Gly Arg Asn Ser Phe Glu Val Arg Val Cys Ala
1 5 10 15
Cys Pro Gly Arg Asp Arg Arg Thr Glu Glu Glu Asn Leu Arg Lys Lys
              20 25 30
Gly Glu Pro His His Glu Leu Pro Pro Gly Ser Thr Lys Arg Ala Leu
        35 40 45
Pro Asn Asn Thr Ser Ser Ser Pro Gln Pro Lys Lys Lys Pro Leu Asp
      50 55 60
Gly Glu Tyr Phe Thr Leu Gln Ile Arg Gly Arg Glu Arg Phe Glu Met
65 70 75 80
Phe Arg Glu Leu Asn Glu Ala Leu Glu Leu Lys Asp Ala Gln Ala Gly
                  85 90 95
Lys Glu Pro Gly Gly Ser Arg Ala His Ser Ser His Leu Lys Ser
            100 105 110
SEQ ID NO: 5
Thr Leu Glu Asp Ser Ser Gly Asn Leu Leu Gly Arg Asn Ser Phe Glu
1 5 10 15
Val Arg Val Cys Ala Cys Pro Gly Arg Asp Arg Arg Thr Glu Glu Glu
              20 25 30
Asn Leu Arg Lys Lys Gly Glu Pro His His Glu Leu Pro Pro Gly Ser
        35 40 45
Thr Lys Arg Ala Leu Pro Asn Asn Thr Ser Ser Ser Pro Gln Pro Lys
    50 55 60
Lys Lys Pro Leu Asp Gly Glu Tyr Phe Thr Leu Gln Ile Arg Gly Arg
65 70 75 80
Glu Arg Phe Glu Met Phe Arg Glu Leu Asn Glu Ala Leu Glu Leu Lys
                85 90 95
Asp Ala Gln Ala Gly Lys Glu Pro Gly Gly Ser Arg Ala His Ser Ser
            100 105 110
His Leu Lys Ser
          115
SEQ ID NO: 6
Met Glu Glu Pro Gln Ser Asp Pro Ser Val Glu Pro Pro Leu Ser Gln
1 5 10 15
Glu Thr Phe Ser Asp Leu Trp Lys Leu Leu Pro Glu Asn Asn Val Leu
            20 25 30
Ser Pro Leu Pro Ser Gln Ala Met Asp Asp Leu Met Leu Ser Pro Asp
        35 40 45
Asp Ile Glu Gln Trp Phe Thr Glu Asp Pro Gly Pro Asp Glu Ala Pro
      50 55 60
Arg Met Pro Glu Ala Ala Pro Pro Val Ala Pro Ala Pro Ala Ala Pro
65 70 75 80
Thr Pro Ala Ala Pro Ala Pro Ala Pro Ser Trp Pro Leu Ser Ser Ser
                    85 90 95
Val Pro Ser Gln Lys Thr Tyr Gln Gly Ser Tyr Gly Phe Arg Leu Gly
              100 105 110
Phe Leu His Ser Gly Thr Ala Lys Ser Val Thr Cys Thr Tyr Ser Pro
          115 120 125
Ala Leu Asn Lys Met Phe Cys Gln Leu Ala Lys Thr Cys Pro Val Gln
    130 135 140
Leu Trp Val Asp Ser Thr Pro Pro Pro Gly Thr Arg Val Arg Ala Met
145 150 155 160
Ala Ile Tyr Lys Gln Ser Gln His Met Thr Glu Val Val Arg Arg Cys
                  165 170 175
Pro His His Glu Arg Cys Ser Asp Ser Asp Gly Leu Ala Pro Pro Gln
                180 185 190
His Leu Ile Arg Val Glu Gly Asn Leu Arg Val Glu Tyr Leu Asp Asp
        195 200 205
Arg Asn Thr Phe Arg His Ser Val Val Val Pro Tyr Glu Pro Pro Glu
    210 215 220
Val Gly Ser Asp Cys Thr Thr Ile His Tyr Asn Tyr Met Cys Asn Ser
225 230 235 240
Ser Cys Met Gly Gly Met Asn Arg Arg Pro Ile Leu Thr Ile Ile Thr
                245 250 255
Leu Glu Asp Ser Ser Gly Asn Leu Leu Gly Arg Asn Ser Phe Glu Val
              260 265 270
Arg Val Cys Ala Cys Pro Gly Arg Asp Arg Arg Thr Glu Glu Glu Asn
        275 280 285
Leu Arg Lys Lys Gly Glu Pro His His Glu Leu Pro Pro Gly Ser Thr
    290 295 300
Lys Arg Ala Leu Pro Asn Asn Thr Ser Ser Ser Pro Gln Pro Lys Lys
305 310 315 320
Lys Pro Leu Asp Gly Glu Tyr Phe Thr Leu Gln Ile Arg Gly Arg Glu
                    325 330 335
Arg
SEQ ID NO: 7
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
                20 25 30
Val Met His Trp Val Lys Gln Lys Pro Gly Gln Gly Leu Glu Trp Ile
        35 40 45
Gly Tyr Ile Asn Pro Tyr Asn Asp Gly Thr Lys Tyr Asn Glu Lys Phe
    50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                  85 90 95
Ala Arg Gly Gly Tyr Tyr Ala Met Asp Tyr Trp Gly Gln Gly Thr Ser
              100 105 110
Val Thr Val Ser Ser Glu Ser Gln Ser Phe Pro Asn Val Phe Pro Leu
          115 120 125
Val Ser Cys Glu Ser Pro Leu Ser Asp Lys Asn Leu Val Ala Met Gly
      130 135 140
Cys Leu Ala Arg Asp Phe Leu Pro Ser Thr Ile Ser Phe Thr Trp Asn
145 150 155 160
Tyr Gln Asn Asn Thr Glu Val Ile Gln Gly Ile Arg Thr Phe Pro Thr
                  165 170 175
Leu Arg Thr Gly Gly Lys Tyr Leu Ala Thr Ser Gln Val Leu Leu Ser
            180 185 190
Pro Lys Ser Ile Leu Glu Gly Ser Asp Glu Tyr Leu Val Cys Lys Ile
          195 200 205
His Tyr Gly Gly Lys Asn Arg Asp Leu His Val Pro Ile Pro Ala Val
    210 215 220
Ala Glu Met Asn Pro Asn Val Asn Val Phe Val Pro Pro Arg Asp Gly
225 230 235 240
Phe Ser Gly Pro Ala Pro Arg Lys Ser Lys Leu Ile Cys Glu Ala Thr
                    245 250 255
Asn Phe Thr Pro Lys Pro Ile Thr Val Ser Trp Leu Lys Asp Gly Lys
            260 265 270
Leu Val Glu Ser Gly Phe Thr Thr Asp Pro Val Thr Ile Glu Asn Lys
        275 280 285
Gly Ser Thr Pro Gln Thr Tyr Lys Val Ile Ser Thr Leu Thr Ile Ser
    290 295 300
Glu Ile Asp Trp Leu Asn Leu Asn Val Tyr Thr Cys Arg Val Asp His
305 310 315 320
Arg Gly Leu Thr Phe Leu Lys Asn Val Ser Ser Thr Cys Ala Ala Ser
                  325 330 335
Pro Ser Thr Asp Ile Leu Thr Phe Thr Ile Pro Pro Ser Phe Ala Asp
                340 345 350
Ile Phe Leu Ser Lys Ser Ala Asn Leu Thr Cys Leu Val Ser Asn Leu
          355 360 365
Ala Thr Tyr Glu Thr Leu Asn Ile Ser Trp Ala Ser Gln Ser Gly Glu
    370 375 380
Pro Leu Glu Thr Lys Ile Lys Ile Met Glu Ser His Pro Asn Gly Thr
385 390 395 400
Phe Ser Ala Lys Gly Val Ala Ser Val Cys Val Glu Asp Trp Asn Asn
                  405 410 415
Arg Lys Glu Phe Val Cys Thr Val Thr His Arg Asp Leu Pro Ser Pro
            420 425 430
Gln Lys Lys Phe Ile Ser Lys Pro Asn Glu Val His Lys His Pro Pro
        435 440 445
Ala Val Tyr Leu Leu Pro Pro Ala Arg Glu Gln Leu Asn Leu Arg Glu
    450 455 460
Ser Ala Thr Val Thr Cys Leu Val Lys Gly Phe Ser Pro Ala Asp Ile
465 470 475 480
Ser Val Gln Trp Leu Gln Arg Gly Gln Leu Leu Pro Gln Glu Lys Tyr
                    485 490 495
Val Thr Ser Ala Pro Met Pro Glu Pro Gly Ala Pro Gly Phe Tyr Phe
                    500 505 510
Thr His Ser Ile Leu Thr Val Thr Glu Glu Glu Trp Asn Ser Gly Glu
        515 520 525
Thr Tyr Thr Cys Val Val Gly His Glu Ala Leu Pro His Leu Val Thr
    530 535 540
Glu Arg Thr Val Asp Lys Ser Thr Gly Lys Pro Thr Leu Tyr Asn Val
545 550 555 560
Ser Leu Ile Met Ser Asp Thr Gly Gly Thr Cys Tyr
            565 570
SEQ ID NO: 8
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
            20 25 30
Leu Asn Trp Tyr Gln Gln Lys Pro Asp Gly Thr Val Lys Leu Leu Ile
          35 40 45
Tyr Tyr Thr Ser Arg Leu His Ser Gly Val Pro Ser Arg Phe Ser Gly
    50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                85 90 95
Thr Phe Gly Gly Gly Thr Lys Leu Glu Ile Lys Arg Ala Asp Ala Ala
              100 105 110
Pro Thr Val Ser Ile Phe Pro Pro Ser Ser Glu Gln Leu Thr Ser Gly
        115 120 125
Gly Ala Ser Val Val Cys Phe Leu Asn Asn Phe Tyr Pro Lys Asp Ile
    130 135 140
Asn Val Lys Trp Lys Ile Asp Gly Ser Glu Arg Gln Asn Gly Val Leu
145 150 155 160
Asn Ser Trp Thr Asp Gln Asp Ser Lys Asp Ser Thr Tyr Ser Met Ser
                  165 170 175
Ser Thr Leu Thr Leu Thr Lys Asp Glu Tyr Glu Arg His Asn Ser Tyr
                180 185 190
Thr Cys Glu Ala Thr His Lys Thr Ser Thr Ser Pro lie Val Lys Ser
        195 200 205
Phe Asn Arg Asn Glu Cys
    210
SEQ ID NO: 9
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
              20 25 30
Val Met His Trp Val Lys Gln Lys Pro Gly Gln Gly Leu Glu Trp Ile
          35 40 45
Gly Tyr Ile Asn Pro Tyr Asn Asp Gly Thr Lys Tyr Asn Glu Lys Phe
    50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                  85 90 95
Ala Arg Gly Gly Tyr Tyr Ala Met Asp Tyr Trp Gly Gln Gly Thr Ser
            100 105 110
Val Thr Val Ser Ser
        115
SEQ ID NO: 10
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
            20 25 30
Leu Asn Trp Tyr Gln Gln Lys Pro Asp Gly Thr Val Lys Leu Leu Ile
        35 40 45
Tyr Tyr Thr Ser Arg Leu His Ser Gly Val Pro Ser Arg Phe Ser Gly
      50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                  85 90 95
Thr Phe Gly Gly Gly Thr Lys Leu Glu Ile Lys
              100 105
SEQ ID NO: 11
Ser Tyr Val Met His
1 5
SEQ ID NO: 12
Tyr Ile Asn Pro Tyr Asn Asp Gly Thr Lys Tyr Asn Glu Lys Phe Lys
1 5 10 15
Gly
SEQ ID NO: 13
Gly Gly Tyr Tyr Ala Met Asp Tyr
1 5
SEQ ID NO: 14
Arg Ala Ser Gln Asp Ile Ser Asn Tyr Leu Asn
1 5 10
SEQ ID NO: 15
Tyr Thr Ser Arg Leu His Ser
1 5
SEQ ID NO: 16
Gln Gln Gly Asn Thr Leu Pro Tyr Thr
1 5
```

1. An in vitro or ex vivo method to differentiate Alzheimer's Disease (AD) from Cognitive Unimpaired (CU) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12,
wherein AD is indicated by:
(a) the presence of a truncation of the region denoted by amino acids 1-248 of p53 protein (SEQ ID NO:1),
(b) the presence of at least one of PTMs-1, 3, 4, 5 and 6, and
(c) the absence of PTM-7;
and
wherein CU is indicated by:
(a) the presence of PTM-7,
(b) no truncation of the region denoted by amino acids 1-248 of p53 protein (SEQ ID NO:1), and
(c) the absence of at least one of PTMs-1, 3, 4, 5, and 6,
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

2. An in vitro or ex vivo method to differentiate Alzheimer's Disease (AD) and a neurodegenerative disease selected from the following: Mild Cognitive Impairment (MCI), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein AD is differentiated from a said neurodegenerative diseases as follows (A)-(D), respectively:
(A) AD differentiated from MCI:
wherein AD is indicated by:
(a) a truncation of the region denoted by amino acids 1-248 of p53 protein (SEQ ID NO:1), and
(b) the presence of at least one of PTMs-2, 3, 4, 5, and 6, and
(c) the absence of at least one of PTMs-7 and 10;
and wherein MCI is indicated by:
(a) the presence of at least one of PTMs-7 and 10, and
(b) no truncation of the region denoted by amino acids 1-248 of p53 protein (SEQ ID NO:1), and
(c) the absence of at least one of PTMs-2, 3, 4, 5, and 6;
(B) AD differentiated from FTD:
wherein AD is indicated by the presence of:
(a) a truncation of the region denoted by amino acids 1-248 of p53 protein (SEQ ID NO:1), and
(b) the presence of at least one of PTMs-1, 2, 3, 4, 6 and 11, and
(c) the absence of PTM-9;
wherein FTD is indicated by:
(a) the presence of PTM 9,
(b) no truncation of region denoted by amino acids 1-248 of p53 protein (SEQ ID NO:1), and
(c) the absence of at least one of PTMs-1, 2, 3, 4, 6 and 11;
(C) AD differentiated from LB:
wherein AD is indicated by the presence of:
(a) a truncation of the region denoted by amino acids 1-248 of p53 protein (SEQ ID NO:1), and
(b) the presence of at least one of PTMs-1, 3, 4 and 11;
wherein LB is indicated by:
(a) no truncation of the region denoted by amino acids 1-248 of p53 protein (SEQ ID NO:1), and
(b) the absence of at least one of PTMs-1, 3, 4, and 11;
and
(D) AD differentiated from VD:
wherein AD is indicated by the presence of:
(a) a truncation of the region denoted by amino acids 1-248 of p53 protein (SEQ ID NO:1), and
(b) the presence of at least one of PTMs-1, 3, 4 and 11;
wherein VD is indicated by:
(a) no truncation of the region denoted by amino acids 1-248 of p53 protein (SEQ ID NO:1), and
(b) the absence of at least one of PTMs 1, 2, 3, 6 and 11,
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of
SEQ ID NO: 1.

3. An in vitro or ex vivo method to differentiate Cognitive Unimpaired (CU) and a neurodegenerative disease selected from the following: Mild Cognitive Impairment (MCI), MCI with a prognosis of cognitive decline of AD (MCI to AD), cognitive decline of an asymptomatic AD (CU to AD), Alzheimer's disease (AD), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein CU is differentiated from a said neurodegenerative diseases as follows (A)-(G), respectively:
(A) CU is differentiated from MCI:
wherein CU is indicated by:
(a) the presence of PTM-2,
(b) the absence of at least one of PTM1 and PTM10;
and wherein MCI is indicated by:
(a) the presence of at least one of PTM-1, and PTM-10, and
(b) the absence of PTM-2;
(B) CU is differentiated from MCI to AD:
wherein CU is indicated by:
(a) the presence of PTM-2, and
(b) the absence of at least one of PTM-1, PTM-3, PTM-5, PTM6, and PTM-10;
and wherein MCI to AD is indicated by:
(a) the presence of at least one of PTM-1, PTM-3, PTM-5, PTM-6 and PTM-10, and
(b) the absence of PTM-2
(C) CU is differentiated from CU to AD:
wherein CU is indicated by:
(a) the presence of at least one of PTM-2, 7, 8, and 11
(b) the absence of at least one of PTM-4 and 5,
(c) no truncation of the region denoted by amino acids 1-248 of p53 protein (SEQ ID NO:1)
wherein CU to AD is indicated by:
(a) the presence of at least one of PTM-4, and PTM-5,
(b) the absence of at least one of PTM-2, PTM-7, PTM-8 and PTM-11;
(D) wherein CU is differentiated from AD:
wherein CU is indicated by:
(a) the presence of PTM-7,
(b) the absence of PTM-1 and PTM3-6, and
(c) no truncation of the region denoted by amino acids 1-248 of p53 protein (SEQ ID NO:1)
wherein AD is indicated by:
(a) the presence of at least one of PTM-1, and PTM3-6,
(b) the absence of PTM-7, and
(c) truncation of the region denoted by amino acids 1-248 of p53 protein (SEQ ID NO:1);
(E) wherein CU is differentiated from FTD:
wherein CU is indicated by:
(a) the presence of at least one of PTM-2, PTM-7 and 11,
(b) the absence of at least one of PTM-5 and PTM-9;
wherein FTD is indicated by:
(a) the presence of at least one of PTM-5 and PTM-9, and
(b) the absence of at least one of PTM-2, PTM-7 and PTM-11;
(F) wherein CU is differentiated from LB:
wherein CU is indicated by:
(a) the presence of at least one of PTM-7 and PTM-11, and
(b) the absence of at least one of PTM-5 and PTM-6;
wherein LB is indicated by:
(a) the presence of at least one of PTM-5 and PTM-6, and
(b) the absence of at least one of PTM-7 and PTM-11;
(G) wherein CU is differentiated from VD:
wherein CU is indicated by:
(a) the presence of at least one of PTM-2, PTM-7 and PTM-11, and
(b) the absence of at least one of PTM-4 and PTM-5;
wherein VD is indicated by:
(a) the presence of at least one of PTM-4 and PTM-5, and
(b) the absence of at least one of PTM-2, PTM-7 and PTM-11;
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

4. An in vitro or ex vivo method to differentiate Mild Cognitive Impairment (MCI) from a neurodegenerative disease selected from the following:, MCI with a prognosis of cognitive decline of AD (MCI to AD), cognitive decline of an asymptomatic subject to AD (CU to AD), Alzheimer's disease (AD), Frontotemporal dementia (FTD), Lewi's body dementia (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein MCI is differentiated from a said neurodegenerative disease as follows (A)-(F):
(A) MCI is differentiated from MCI progression to AD:
wherein MCI is indicated by:
(a) the absence of at least one of PTM-3, PTM-5 and PTM-6,
wherein MCI progression to AD is indicated by:
(a) the presence of at least one of PTM-3, PTM-5 and PTM-6;
(B) MCI is differentiated from CU progression to AD:
wherein MCI is indicated by:
(a) the presence of at least one of PTM-1, PTM-7, PTM-10 and PTM-11, and
(b) the absence of at least one of PTM-4, PTM-5 and PTM-9;
wherein CU progression to AD is indicated by:
(a) the presence of at least one of PTM-4, PTM-5 and PTM-9,
(b) the absence of at least one of PTM-1, PTM-7, PTM-10 and PTM-11
(C) MCI is differentiated from AD:
wherein MCI is indicated by:
(a) the presence of at least one of PTM-7, PTM-8 and PTM-10,
(b) the absence of at least one of PTM-2 to PTM-6, and
(c) no truncation of the region denoted by amino acids 1-248 of p53 protein (SEQ ID NO:1);
wherein AD is indicated by:
(a) the presence of at least one of PTM-2 to PTM-6,
(b) the absence of at least one of PTM-7, PTM-8 and PTM-10, and
(c) truncation of the region denoted by amino acids 1-248 of p53 protein (SEQ ID NO:1);
(D) MCI is differentiated from FTD:
wherein MCI is indicated by:
(a) the presence of at least one of PTM-1, PTM7 , PTM-10 and PTM-11
(b) the absence of at least one of PTM-5 and PTM-9;
wherein FTD is indicated by:
(a) the presence of at least one of PTM-5 and PTM-9, and
(b) the absence of at least one of PTM-1, PTM7 , PTM-10 and PTM-11;
(E) MCI is differentiated from LB:
wherein MCI is indicated by:
(a) the presence of at least one of PTM-1, PTM-7, PTM-10 and PTM-11,
(b) the absence of at least one of PTM-2, and PTM-4 to PTM-6
wherein LB is indicated by:
(a) the presence of at least one of PTM-2, PTM-4, PTM5 and PTM-6
(b) the absence of at least one of PTM-1, PTM-7, PTM-10 and PTM-11;
(F) MCI is differentiated from VD:
wherein MCI is indicated by:
(a) the presence of at least one of PTM-1, PTM7, PTM-10 and PTM-11, and
(b) the absence of at least one of PTM-4 and PTM-5;
wherein VD is indicated by:
(a) the presence of at least one of PTM-5 and PTM-5, and
(b) the absence of at least one of PTM-1, PTM-7, pTM-10 and PTM-11;
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

5. An in vitro or ex vivo method to differentiate cognitive decline of an asymptomatic subject to AD (CU to AD) from a neurodegenerative disease selected from the following:, MCI with a prognosis of cognitive decline of AD (MCI to AD), Alzheimer's disease (AD), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein CU is differentiated from a said neurodegenerative disease as follows (A)-(E):
to (A) CU progression to AD is differentiated from MCI progression to AD:
wherein CU progression to AD is indicated by:
(a) the presence of at least one of PTM-4, and PTM-9, and
(b) the absence of at least one of PTM-1, PTM-3, PTM-6, PTM-7, PTM-10 and PTM-11;
wherein MCI progression to AD is indicated by:
(a) the presence of at least one of PTM-1, PTM-3, PTM-6, PTM-7, PTM-10 and PTM-11, and
(b) the absence of at least one of PTM-4 and PTM-9;
(B) CU progression to AD is differentiated from AD:
wherein CU progression to AD is indicated by:
(a) the presence of PTM-9,
(b) the absence of at least one of PTM-1, PTM-2, PTM-3, PTM-6, and PTM-11, and
(c) no truncation of the region denoted by amino acids 1-248 of p53 protein (SEQ ID NO:1);
wherein AD is indicated by:
(a) the presence of at least one of PTM-1, PTM-2, PTM-3, PTM-6 and PTM-11, and
(b) the absence of PTM-9, and
(c) truncation of the region denoted by amino acids 1-248 of p53 protein (SEQ ID NO:1);
(C) CU progression to AD is differentiated from FTD:
wherein CU progression to AD is indicated by:
(a) the presence of PTM-4;
wherein FTD is indicated by:
(a) the absence of PTM-4;
(D) CU progression to AD is differentiated from LB:
wherein CU progression to AD is indicated by:
(a) the presence of at least one of PTM-4 and PTM-9, and
(b) the absence of at least one of PTM-2 and PTM-6;
wherein LB is indicated by:
(a) the presence of at least one of PTM-2, and PTM-6, and
(b) the absence of at least one of PTM-4 and PTM-9;
(E) CU progression to AD is differentiated from VD:
wherein CU progression to AD is indicated by:
(a) the presence of PTM-9;
wherein VD is indicated by:
(a) the absence of PTM-9;
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

6. An in vitro or ex vivo method to differentiate Mild Cognitive Impairment with a prognosis of cognitive decline of AD (MCI progression to AD) from a neurodegenerative disease selected from the following:, cognitive decline of an asymptomatic subject to AD (CU progression to AD), Alzheimer's disease (AD), Frontotemporal dementia (FTD), Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (U-p53), said PTMs comprising PTMs-1-12, wherein MCI is differentiated from a said neurodegenerative disease as follows (A)-(E):
(A) MCI progression to AD is differentiated from CU progression to AD:
wherein MCI progression to AD is indicated by:
(c) the presence of at least one of PTM-1, PTM-3, PTM-6, PTM-7, PTM-10 and PTM-11, and
(d) the absence of at least one of PTM-4 and PTM-9;
wherein CU progression to AD is indicated by:
(c) the presence of at least one of PTM-4, and PTM-9, and
(d) the absence of at least one of PTM-1, PTM-3, PTM-6, PTM-7, PTM-10 and PTM-11;
(B) MCI progression to AD is differentiated from AD:
wherein MCI progression to AD is indicated by:
(d) the presence of at least one of PTM-7 and PTM-10,
(e) the absence of at least one of PTM-2, and PTM-4, and
(f) no truncation of the region denoted by amino acids 1-248 of p53 protein (SEQ ID NO:1);
wherein AD is indicated by:
(d) the presence of at least one of PTM-2 and PTM-4,
(e) the absence of at least one of PTM-7 and PTM-10, and
(f) truncation of the region denoted by amino acids 1-248 of p53 protein (SEQ ID NO:1);
(C) MCI progression to AD is differentiated from FTD:
wherein MCI progression to AD is indicated by:
(c) the presence of at least one of PTM-1, PTM-3, PTM-6, PTM-7, PTM-10 and PTM-11, and
(d) the absence of PTM-9;
wherein FTD is indicated by:
(c) the presence of PTM-9, and
(d) the absence of at least one of PTM-1, PTM-3, PTM-6, PTM-7, PTM-10 and PTM-11;
(D) MCI progression to AD is differentiated from LB:
wherein MCI progression to AD is indicated by:
(c) the presence of at least one of PTM-1, PTM-3, PTM-7, PTM-10 and PTM-11, and
(d) the absence of PTM-2;
wherein LB is indicated by:
(c) the presence of PTM-2, and
(d) the absence of PTM-1, PTM-3, PTM-7, PTM-10 and PTM-11;
(E) MCI progression to AD is differentiated from VD:
wherein MCI progression to AD is indicated by:
(c) the presence of at least one of PTM-1, PTM-3, PTM-6, PTM-7, PTM-10 and PTM-11, and
(d) the absence of PTM-4;
wherein VD is indicated by:
(c) the presence of PTM-4, and
(d) the absence of at least one of PTM-1, PTM-3, PTM-6, PTM-7, PTM-10 and PTM-11;
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

7. An in vitro or ex vivo method to differentiate Frontotemporal dementia (FTD) from a neurodegenerative disease selected from Lewi's body (LB) and Vascular dementia (VD) in a subject, the method comprising detecting in a biofluid sample of said subject the presence and/or absence of one or more post-translation modifications (PTMs) in the region of amino acids 1-371 of the p53 protein (SEQ ID NO:1), said PTMs comprising PTMs-1-12, wherein FTD is differentiated from LB and from VD as follows (A) and (B), respectively:
(A) FTD is differentiated from LB:
wherein FTD is indicated by:
(a) the presence of PTM-9, and
(b) the absence of at least one of PTM 2 and PTM-6;
wherein LB is indicated by:
(c) the presence of at least one of PTM 2 and PTM-6, and
(d) the absence of PTM-9;
(B) FTD is differentiated from VD:
wherein FTD is indicated by:
(a) the presence of PTM-9, and
(b) the absence of PTM-4;
wherein VD is indicated by:
(c) the presence of PTM-4, and
(d) the absence of PTM-9;
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

8. An in vitro or ex vivo method to differentiate Lewi's body dementia (LB) and Vascular dementia (VD) in a subject, the method comprising detecting in a biofluid sample of said subject the presence and/or absence of one or more post-translation modifications (PTMs) in the region of amino acids 1-371 of the p53 protein (SEQ ID NO:1), said PTMs comprising PTMs-1-12,
wherein LB and VD are differentiated by:
wherein LB is indicated by:
(a) the presence of at least one of PTM 2 and PTM-6, and
(b) the absence of PTM-4;
and
wherein VD is indicated by:
(a) the presence of PTM-4, and
(b) the absence of PTM-2 and PTM-6;
wherein said PTMs 1-12 comprise:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

9. An in vitro or ex vivo method to identify in a subject a neurodegenerative disease selected from: Mild Cognitive Impairment (MCI), Frontotemporal dementia (FTD), Lewi's body (LB) and Vascular dementia (VD), cognitively unimpaired (CU) leading to AD, and MCI leading to AD, or to identify said subject as cognitively unimpaired (CU), wherein said method comprises detecting in a biofluid sample of said subject the presence and/or absence of one or more post-translation modifications (PTMs) in the region of amino acids 1-371 of the p53 protein (U-p53), said PTMs comprising PTMs-1-12, wherein a CU subject or a selected neurodegenerative disease is indicated as set forth in one of (A)-(H), respectively:
(A) wherein a cognitively unimpaired (CU) subject is indicated by:
(a) the presence of PTM-7, and optionally the presence of at least one of PTMs-2 and 8,
(b) no truncation of the region denoted by amino acids 1-248 of p53 protein (SEQ ID NO:1), and
(c) the absence of at least one of PTMs-1, 3, 4, 5, and 6, and optionally the additional absence of PTMs-9 and 10,
thereby identifying AD in said subject;
(B) wherein MCI in said subject is indicated by:
(a) the presence of at least one of PTMs-7 and 10, and optionally the additional presence of at least one of PTMs-a 1, 8 and 11,
(b) no truncation of the region denoted by amino acids 1-248 of p53 protein (SEQ ID NO:1), and
(c) the absence of at least one of PTMs-2, 3, 4, 5, and 6, and optionally the additional absence of PTM-9,
thereby identifying MCI in said subject;
(C) wherein a progression of CU to AD in said subject is indicated by:
(a) the presence of at least one of PTMs-4, 5 and 9,
(b) no truncation of the region denoted by amino acids 1-248 of p53 protein (SEQ ID NO:1), and
(c) the absence of at least one of PTMs-1, 2, 3, 6, 7, 8 and
optionally the additional absence of PTM-10 and 11, thereby identifying the progression of CU to AD in said subject;
(D) wherein a progression of MCI to AD in said subject is indicated by:
(a) the presence of at least one of PTMs-1, 3, 5, 6, 7, 8, and optionally the additional presence of at least one of PTMs-10 and 11,
(b) no truncation of the region denoted by amino acids 1-248 of p53 protein (SEQ ID NO:1), and
(c) the absence of at least one of PTMs-2, 4 and 9, thereby identifying the progression of MCI to AD in said subject;
(E) wherein AD in said subject is indicated by:
(a) the presence of at least one of PTMs-3, 4, 5, and 6, and optionally the additional presence of at least one of PTMs-1, 2, 8 and 11,
(b) a truncation of the region denoted by amino acids 1-248 of p53 protein (SEQ ID NO:1), and
(c) the absence of at least one of PTMs-7 and 9, and optionally the additional absence of PTMs-10, thereby to identify AD in said subject;
(F) wherein FTD in said subject is indicated by:
(a) the presence of at least one of PTMs-5, 7, 8, and 9,
(b) no truncation of the region denoted by amino acids 1-248 of p53 protein (SEQ ID NO:1), and
(c) the absence of at least one of PTMs-1, 2, 3, 4, 6, 10 and 11, thereby to identify FTD in said subject;
(G) wherein LB in said subject is indicated by:
(a) the presence of at least one of PTMs-2, 5, 6, and 8,
(b) no truncation of the region denoted by amino acids 1-248 of p53 protein (SEQ ID NO:1), and
(c) the absence of at least one of PTMs-1, 3, 4, 7, and 9, and optionally the additional absence of at least one of PTMs-10 and 11,
thereby to identify LB in said subject;
and
(H) wherein VD in said subject is indicated by:
(a) the presence of at least one of PTMs-4, 5, and 8,
(b) no truncation of the region denoted by amino acids 1-248 of p53 protein (SEQ ID NO:1), and
(c) the absence of at least one of PTMs-1, 2, 3, .6, 7, and 9, and optionally the additional absence of at least one of PTMs-10 and 11,
thereby to identify VD in said subject;
wherein said PTMs 1-12 of (A)-(H) are defined as:
PTM-1 at the amino acid M1,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33, and
PTM-12 is the truncation of amino acids 1-248 of SEQ ID NO: 1.

10. The in vitro or ex vivo method of claim 1, wherein said biofluid is blood, plasma, serum, saliva, urine, cerebrospinal fluid and neuronal cells, preferably said biofluid is blood, preferably said biofluid is plasma, and preferably said biofluid is cerebrospinal fluid.

11. The in vitro or ex vivo method of claim 1, wherein the p53 protein is captured in a biofluid sample by performing the following steps of:
(i) providing a biofluid sample;
(ii) performing protein immunoprecipitation by an antibody that binds a p53 protein, said antibody comprising heavy chain CDRs 1, 2 and 3 (SEQ ID NOs: 11, 12 and 13, respectively) and light chain CDRs 1, 2 and 3 (SEQ ID NOs: 14, 15 and 16, respectively).;
(iii) performing protein fragmentation by trypsin.

12. The in vitro or ex vivo method of claim 11, wherein said PTMs are detected by HPLC-mass spectrometry and/or Edman degradation.

13. The in vitro or ex vivo method of claim 1, wherein:
the post-translation modification PTM-1 has a group CO—CH₃ branched to the amino acid M1 of the p53 protein;
the post-translation modification PTM-2 has a group CO—CH₃ branched to the amino acid K164 of the p53 protein;
the post-translation modification PTM-3 has a group CO—CH₃ branched to the amino acid K370 of the p53 protein;
the post-translation modification PTM-4 has a ubiquitination site [GG] branched at the amino acid K101 of the p53 protein;
the post-translation modification PTM-5 has a ubiquitination site [GG] branched at the amino acid K120 of the p53 protein;
the post-translation modification PTM-6 has a ubiquitination site [GG] branched at the amino acid K132 of the p53 protein;
the post-translation modification PTM-7 has a ubiquitination site [GG] branched at the amino acid K139 of the p53 protein;
the post-translation modification PTM-8 has a ubiquitination site [GG] branched at the amino acid K291 of the p53 protein;
the post-translation modification PTM-9 has a ubiquitination site [GG] branched at the amino acid K357 of the p53 protein;
the post-translation modification PTM-10 has phosphorylation at the amino acid S6 of the p53 protein;
the post-translation modification PTM-11 has phosphorylation at the amino acid S33 of the p53 protein.

14. An in vitro or ex vivo method for the diagnosis or prognosis of a neurodegenerative disease, the method comprising the step of:
a) analysing a biofluid sample for the presence of post-translation modifications (PTMs) in the region of amino acids 1-371 of the p53 protein (U-p53), said PTMs being:
PTM-1 at the amino acid MI,
PTM-2 at the amino acid K164,
PTM-3 at the amino acid K370,
PTM-4 at the amino acid L101,
PTM-5 at the amino acid K120,
PTM-6 at the amino acid K132,
PTM-7 at the amino acid K139,
PTM-8 at the amino acid K291,
PTM-9 at the amino acid K357,
PTM-10 at the amino acid S6,
PTM-11 at the amino acid S33,
wherein the presence of at least two PTMs selected from PT PTM-7, PTM-8, and PIM-11 is indicative of a cognitive unimpaired subject (CU),
b) identifying in said sample the presence of:
at least two PTMs selected from PIM-1, PTM-3, PTM-4, PTM-5, PTM-6, PTM-9, and PIM-10, and
at least one PTM selected from PTM-2, PTM-7. PTM-8, and PTM-11, as indicative of the occurrence of or the risk of development of a neurological disease, said neurodegenerative disease being selected from Mild Cognitive Impairment (MCI), Alzheimer's disease (AD), Fronto-temporal dementia (FTD), Lewrs Body (LB), and vascular dementia (VD),
c) wherein
the presence of PTM-1, and PTM-10 is indicative of MCI;
the presence of at least two PTMs selected from PTM-4, PTM-5, and PIM-9 is indicative of a prognosis of cognitive decline to AD of an asymptomatic subject;
the presence of at least two PTMs selected from PTM-1. PTM-3, PTM-5, PTM-6, and PTM-10 is indicative of MCI with a prognosis of cognitive decline to AD;
the presence of PIM-5, and PTM-9 is indicative of FTD;
the presence of PTM-5, and PTM-6 is indicative of LB;
the presence of PTM-4, and PTM-5 is indicative of VD,
or wherein said in vitro or ex vivo method is for differentiating Alzheimer's disease, from other neurodegenerative diseases, wherein in step b) AD is indicated by:
a truncation of amino acids 1-248 of the p53 protein (SEQ ID NO: 1), and
the presence of at least two PTMs selected from PTM-1, PTM-3, PTM-4, PTM- 5_{; and PTM-}6, in a residual amount of untruncated sequence;
(preferably, wherein:
the post-translation modification PTM-1 has a group CO—CH₃ branched to the amino acid MI of the p53 protein;
the post-translation modification PTM-2 has a group CO—CH₃ branched to the amino acid K164 of the p53 protein;
the post-translation modification PTM-3 has a group CO—CH₃ branched to the amino acid K370 of the p53 protein;
the post-translation modification PTM-4 has a ubiquitination site [GG] branched at the amino acid K101 of the p53 protein;
the post-translation modification PTM-5 has a ubiquitination site [GG] branched at the amino acid K120 of the p53 protein;
the post-translation modification PTM-6 has a ubiquitination site [GG] branched at the amino acid K132 of the p53 protein;
the post-translation modification PTM-7 has a ubiquitination site [GG] branched at the amino acid K139 of the p53 protein;
the post-translation modification PTM-8 has a ubiquitination site [GG] branched at the amino acid K291 of the p53 protein;
the post-translation modification PTM-9 has a ubiquitination site [GG] branched at the amino acid K357 of the p53 protein;
the post-translation modification PTM-10 has phosphorylation at the amino acid S6 of the p53 protein;
the post-translation modification PTM -11 has phosphorylation at the amino acid S33 of the p53 protein);
and
(preferably, wherein in said step a), the p53 protein is captured in a biofluid sample by performing the following sub-steps of:
(i) providing a biofluid sample;
(ii) performing protein immunoprecipitation by an antibody that binds a p53 protein;
(iii) performing protein fragmentation by trypsin;
and said step b) is performed by HPLC-mass spectrometry and Peptide Mass Fingerprint).

15. The in vitro or ex vivo method of claim 14, said in vitro or ex vivo method being for differentiating Alzheimer's disease from other neurodegenerative diseases, wherein in step b) the AD is indicated by:
a truncation of amino acids 1-248 of SEQ ID NO: 1, and
the presence of all PTM-1, PTM-3, PTM-4, PTM-5, and PTM-6.

16. The in vitro or ex vivo method of claim 14, wherein the presence of all PTM-4, PTM-5, and PTM-9 is indicative of a prognosis of cognitive decline to AD of an asymptomatic subject; and wherein the presence of all PIM-1, PIM-3, PTM-5, PT1 -6, and PTM-10 is indicative of MCI with a prognosis of cognitive decline to AD.

17. The in vitro or ex vivo method of claim 14, wherein the immunoprecipitation of sub- step (ii) is performed with a monoclonal/polyclonal antibody that binds to a p53 peptide, where preferably, said monoclonal antibody is the antibody 2D3A8.

18. The in vitro or ex vivo method of claim 24, wherein the biological sample of step a) is subjected to protein plasma depletion by HPLC or chromatographic columns or chemical treatment, before performing the step (ii).


SEQUENCE LISTING (16 sequences)

SEQ ID NO: 1 - protein, 392 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 2 - protein, 101 aa, Homo sapiens
EVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKS

SEQ ID NO: 3 - protein, 123 aa, Homo sapiens
RPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKS

SEQ ID NO: 4 - protein, 111 aa, Homo sapiens
SGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKS

SEQ ID NO: 5 - protein, 116 aa, Homo sapiens
TLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKS

SEQ ID NO: 6 - protein, 337 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRER

SEQ ID NO: 7 - protein, 572 aa, Mus musculus
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 8 - protein, 214 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 117 aa, Mus musculus
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSS

SEQ ID NO: 10 - protein, 107 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 11 - protein, 5 aa, Mus musculus
SYVMH

SEQ ID NO: 12 - protein, 17 aa, Mus musculus
YINPYNDGTKYNEKFKG

SEQ ID NO: 13 - protein, 8 aa, Mus musculus
GGYYAMDY

SEQ ID NO: 14 - protein, 11 aa, Mus musculus
RASQDISNYLN

SEQ ID NO: 15 - protein, 7 aa, Mus musculus
YTSRLHS

SEQ ID NO: 16 - protein, 9 aa, Mus musculus
QQGNTLPYT